(19) United States
(12) Patent Application Publication
(10) Pub. No.: US 2010/0330113 A1
(43) Pub. Date: Dec. 30, 2010

(54) Vaccine Antigens

(75) Inventors: David Francis Kirke, Slough (GB); Michael James Francis, Amersham (GB)

Correspondence Address: MERCK;PATENT DEPARTMENT (K-6-1, 1990), 2000 GALLOPING HILL ROAD, KENILWORTH, NJ, 07033-0530, US

(73) Assignee: Intervet International B.V.

(21) Appl. No.: 12/763,720

(22) Filed: Apr. 20, 2010

Related U.S. Application Data
(63) Continuation-in-part of application No. 12/338,192, filed on Dec. 18, 2008, now Pat. No. 7,811,583.
(60) Provisional application No. 61/014,782, filed on Dec. 19, 2007.

(51) Int. Cl. A61K 39/02; C07K 14/00; C07K 16/00; C07H 21/04; C12N 15/63; C12N 1/21; C12P 21/02; A61P 31/04; A61P 31/12

(52) U.S. Cl. 424/190.1; 530/350; 530/387.9; 536/23.7; 435/320.1; 435/252.33; 435/69.3

(57) ABSTRACT

The present invention discloses novel proteins, e.g., antigens. The present invention further discloses nucleic acids that encode these proteins. The present invention also discloses the use of the proteins, e.g., antigens, and nucleic acids to prepare vaccines against salmonid rickettsial septicemia (SRS). The present invention also discloses vaccines that can be used to protect fish from Piscirickettsia salmonis, as well as other pathogens. In addition, the present invention discloses methods of using the vaccines of the present invention to protect fish from SRS as well as from other pathogenic diseases.

### CROSS-REFERENCE TO RELATED APPLICATIONS

This application is a continuation-in-part of non-provisional application U.S. Ser. No. 12/338,192, filed Dec. 18, 2008, which claims priority under 35 U.S.C. §119(e) of provisional application U.S. Ser. No. 61/014,782 filed Dec. 19, 2007. Priority is claimed to both earlier applications.

### BACKGROUND OF THE INVENTION

1. Field of Invention

The present invention relates to novel proteins. The present invention also pertains to the nucleic acids that encode these proteins. The present invention further relates to a process of preparing a vaccine against salmonid rickettsial septicemia (SRS) using the proteins as antigens, or the nucleic acids in bacterial hosts to express such antigens. The present invention also relates to bacterins and viral antigens that can be combined to form a vaccine against SRS. The present invention also pertains to vaccines for preventing SRS, as well as preventing other bacterial and/or viral infections in fish.

2. Background

Salmonid rickettsial septicemia (SRS), also known as piscirickettsiosis, is a fatal disease in salmonids. Although the etiological agent for SRS was identified in the late 1980's as Piscirickettsia salmonis, antibiotics proved to be an unsuccessful treatment, due, at least in part, to the intracellular nature of this bacterium [Bravo and Campos, FHS/AFS Newsl. 17:3 (1989); U.K. Patent Application 2 356 632]. As a consequence of the lack of a viable treatment, millions of farmed salmon die of SRS each year just in southern Chile alone [Smith et al., Dis. Aquat. Organ. 37(3):165-172 (1999)]. In addition, recent reports demonstrate a link between Piscirickettsia-like bacteria and disease syndromes in non-salmonid fish [see, Mauel and Miller, Veterin. Microbiol. 87(4):279-289 (2002)].

The Salmonidae family (salmonids) includes salmon, trout, char, and whitefish. Salmonids serve both as a food source and as a game fish. Moreover, in countries such as Chile, Norway, Canada, the United Kingdom, Ireland, and the United States, salmonids have become an important commercial product due, at least in part, to the ability of fish farmers to artificially spawn, incubate and raise the salmonids in captivity.

Unlike fish originating in the wild, those raised in captivity are amenable to prophylactic treatments such as vaccination. So far, several potential vaccines have been described, such as one based on a specific Piscirickettsia salmonis antigen, a 17 kDa lipoprotein OspA [U.K. Patent Application 2 356 632; see also WO 01/68865 A2]. Additional potential vaccines against Piscirickettsia salmonis are described by WO05035558 A2 and WO2006037383 A1, the contents of which are hereby incorporated by reference in their entireties. These published international patent applications describe an isolated Piscirickettsia salmonis ^{Ps}p45 protein and antigenic fragments thereof, as well as other SRS antigens.

The coding sequence for ^{Ps}p45 protein is contained by a recombinant Chilean strain of Yersinia ruckeri that has been deposited (BCCM accession No. LMG P-22044). The nucleotide coding sequence of the ^{Ps}p45 protein within the deposited recombinant Yersinia ruckeri is included within SEQ ID NO: 16. The amino acid sequence of the ^{Ps}p45 protein within the deposited recombinant Yersinia ruckeri is SEQ ID NO: 7 [the amino acid sequence of SEQ ID NO: 8 is identical to that of SEQ ID NO: 7, except SEQ ID NO: 8 lacks the sequence for the signal peptide.]

In addition to Piscirickettsia salmonis, other pathogens are known to cause disease in farmed fish, including salmon. One such pathogen is the Infectious Pancreatic Necrosis virus (IPN virus), which is an unenveloped, icosahedral, bisegmented dsRNA virus. The IPN virus contains one main structural protein, VP2 (52 kDa) and three additional proteins, VP1 (90 kDa), VP3 (30 kDa) and VP4 (28 kDa). VP2 is the main protein of the outer capsid and is therefore immunologically important in recognition and bonding of the virus. VP1 is thought to be a polymerase, whereas VP3 and VP4 are internal proteins. VP4 is believed to correspond to a form of VP3 fragment formed during viral differentiation [see, WO 02/38770 A1, the contents of which are hereby incorporated by reference in their entireties]. Nucleotide and amino acid sequences for VP2 and VP3 have been determined [see, Havarstein et al., J. Gen. Virol. 71:299-308 (1990); Pryde et al., Archives of Vir. 129:287-293 (1992)].

There, therefore remains a need to provide new safe and effective vaccines against Piscirickettsia salmonis. In addition, there remains a need to identify new antigens that can be used in such vaccines. Furthermore, there is a need to obtain nucleic acids that encode such antigens. In addition, there is a need to provide methods of vaccinating fish to protect them from Piscirickettsia salmonis and Piscirickettsia-like bacteria. Furthermore, there is a need to provide vaccines that can protect fish against Piscirickettsia salmonis and other unrelated pathogens, particularly those of commercial importance, such as the IPN virus.

The citation of any reference herein should not be construed as an admission that such reference is available as “prior art” to the instant application.

### SUMMARY OF THE INVENTION

The present invention provides isolated, recombinant, or both isolated and recombinant proteins, as well as antigenic fragments thereof. One such protein is p₁90, a 90 kDa protein (ORF1), as described below. Another 90 kDa protein is p₂90, (ORF 2) as described below. p₁90 and p₂90 can be expressed from any suitable nucleic acid that encodes one or both of them, respectively, e.g., either DNA or RNA. In addition, p₁90 or p₂90 can be used as antigens in vaccines against SRS, either alone, or in combination with each other and/or other antigens.

The present invention further provides nucleic acids that encode the isolated and/or recombinant proteins and/or antigenic fragments of the proteins. Furthermore, the present invention provides nucleotide probes and PCR primers that can be used, e.g., to identify such nucleic acids that encode these proteins. In addition, the present invention provides recombinant vectors that encode the proteins of the present invention, or fragments thereof, such as recombinant viruses and bacteria. Corresponding attenuated or killed recombinant bacteria, e.g., bacterins prepared from the recombant bacterial vectors are also provided.

The present invention further provides vaccines that comprise proteins of the present invention and/or antigenic fragments of these antigens. These antigens may be placed into a vaccine in any number of forms including as a recombinant protein itself, and/or as a recombinant protein expressed by a recombinant vector such as a recombinant gram negative bacterium, or as a naked DNA. In a particular embodiment of the present invention, the recombinant gram negative bacterium is a recombinant E. coli cell.

Preferably the recipient of a vaccine of the present invention receives protection from Piscirickettsia salmonis. In one embodiment, the vaccine comprises recombinant E. coli that encode and express the p₁90 and/or p₂90 proteins and/or antigenic fragments of the p₁90 and/or p₂90 protein. In a particular embodiment of this type, such recombinant E. coli are inactivated prior to being added to the vaccine and/or prior to the administration of the vaccine to the animal subject. In addition, booster vaccines are also provided by the present invention.

Antibodies that bind to the proteins of the present invention are also provided. Such antibodies can be used: to demonstrate the presence of, identify, and/or purify the proteins of the present invention.

Accordingly, the present invention provides a p₁90 protein that comprises an amino acid sequence comprising at least 60% identity with the amino acid sequence of SEQ ID NO: 2. In a particular embodiment of this type the p₁90 protein comprises an amino acid sequence comprising at least 75% identity with the amino acid sequence of SEQ ID NO: 2. In another embodiment of this type the p₁90 protein comprises an amino acid sequence comprising at least 90% identity with the amino acid sequence of SEQ ID NO: 2. In still another embodiment of this type the p₁90 protein comprises an amino acid sequence comprising at least 95% identity with the amino acid sequence of SEQ ID NO: 2.

In yet another embodiment of this type the p₁90 protein comprises an amino acid sequence comprising SEQ ID NO: 2 that comprises one or more conservative amino acid substitutions. In still another embodiment the ^{p}₁90 protein comprises an amino acid sequence comprising SEQ ID NO: 2 that comprises one to ten amino acid substitutions. In a particular embodiment of this type the p₁90 protein comprises an amino acid sequence of SEQ ID NO: 2 that comprises one to ten conservative amino acid substitutions. In a specific embodiment, the p₁90 protein comprises the amino acid sequence of SEQ ID NO: 2. In another embodiment, the p₁90 protein consists essentially of the amino acid sequence of SEQ ID NO: 2. P. salmonis variants of the p₁90 protein that comprises the amino acid sequence of SEQ ID NO: 2 are also included as part of the present invention.

Preferably, all of the p₁90 proteins of the present invention bind to an antibody elicited by the p₁90 protein that has the amino acid sequence of SEQ ID NO: 2. More preferably, when a p₁90 protein of the present invention is included as an antigen in a vaccine administered to salmonids, the vaccine provides protection against SRS to the vaccinated salmonids.

The present invention also provides a p₂90 protein that comprises an amino acid sequence comprising at least 60% identity with the amino acid sequence of SEQ ID NO: 4. In a particular embodiment of this type the p₂90 protein comprises an amino acid sequence comprising at least 75% identity with the amino acid sequence of SEQ ID NO: 4. In another embodiment of this type the p₂90 protein comprises an amino acid sequence comprising at least 90% identity with the amino acid sequence of SEQ ID NO: 4. In still another embodiment of this type the p₂90 protein comprises an amino acid sequence comprising at least 95% identity with the amino acid sequence of SEQ ID NO: 4.

In yet another embodiment of this type the p₂90 protein comprises an amino acid sequence comprising SEQ ID NO: 4 that comprises one or more conservative amino acid substitutions. In still another embodiment the p₂90 protein comprises an amino acid sequence comprising SEQ ID NO: 4 that comprises one to ten amino acid substitutions. In a particular embodiment of this type the p₂90 protein comprises an amino acid sequence of SEQ ID NO: 4 that comprises one to ten conservative amino acid substitutions. In a specific embodiment, the p₂90 protein comprises the amino acid sequence of SEQ ID NO: 4. In another embodiment, the p₂90 protein consists essentially of the amino acid sequence of SEQ ID NO: 4. Variants of the p₂90 protein that comprises the amino acid sequence of SEQ ID NO: 4 are also included as part of the present invention.

Preferably, all of the p₂90 proteins of the present invention bind to an antibody elicited by the p₂90 protein that has the amino acid sequence of SEQ ID NO: 4. More preferably, when a p₂90 protein of the present invention is included as an antigen in a vaccine administered to salmonids, the vaccine provides protection against SRS to the vaccinated salmonids.

The present invention also provides antigenic fragments of all of the P. salmonis proteins of the present invention. In a particular embodiment, the antigenic fragment is of the p₁90 protein having the amino acid sequence of SEQ ID NO: 2. In another particular embodiment, the antigenic fragment is of the p₂90 protein having the amino acid sequence of SEQ ID NO: 4. The antigenic fragments of the present invention can be in any form including but not limited to: isolated, recombinant, chemically synthesized, both recombinant and isolated, or both chemically synthesized and isolated.

The present invention further provides chimeric polypeptides that comprise a p₁90 protein of the present invention or antigenic fragment thereof, and/or a p₂90 protein of the present invention or antigenic fragment thereof. In one such embodiment, the chimeric polypeptide comprises the p₁90 protein having the amino acid sequence of SEQ ID NO: 2. In an alternative embodiment, the chimeric polypeptide comprises the p₂90 protein having the amino acid sequence of SEQ ID NO: 4. In another embodiment, the chimeric polypeptide comprises an antigenic fragment of a p₁90 protein of the present invention that has the amino acid sequence of SEQ ID NO: 2. In still another embodiment, the chimeric polypeptide comprises an antigenic fragment of the p₂90 protein that comprises the amino acid sequence of SEQ ID NO: 4. The chimeric polypeptides of the present invention can be in any form including but not limited to: isolated, recombinant, chemically synthesized, both recombinant and isolated, or both chemically synthesized and isolated.

The present invention further provides antibodies elicited by the p₁90 or p₂90 proteins of the present invention, including those elicited by a chimeric polypeptide of the present invention. In one embodiment, the antibody is solicited by the p₁90 protein having the amino acid sequence of SEQ ID NO: 2. In an alternative embodiment, the antibody is solicited by the p₂90 protein having the amino acid sequence of SEQ ID NO: 4.

The present invention also provides antibodies solicited by an antigenic fragment of a p₁90 and/or a p₂90 protein of the present invention. In one such embodiment, the antibody is solicited by an antigenic fragment of a p₁90 protein of the present invention having the amino acid sequence of SEQ ID NO: 2. In another embodiment, the antibody is solicited by an antigenic fragment of the p₂90 protein having the amino acid sequence of SEQ ID NO: 4.

In another aspect of the present invention, nucleic acids are provided which encode: the p₁90 proteins, the p₂90 proteins, the antigenic fragments of these p₁90 proteins and/or p₂90 proteins, and/or the corresponding chimeric polypeptides of the present invention. Any of these nucleic acids can further comprise heterologous nucleotide sequences. The nucleic acids of the present is invention can be in any form including but not limited to: isolated, recombinant, chemically synthesized, both recombinant and isolated, or both chemically synthesized and isolated.

In a particular embodiment, a nucleic acid of the present invention encodes a p₁90 protein that comprises the amino acid sequence of SEQ ID NO: 2. In a particular embodiment of this type the nucleic acid comprises the nucleotide sequence of SEQ ID NO: 1. In an alternative embodiment the nucleic acid comprises the nucleotide sequence of SEQ ID NO: 5.

In another embodiment, a nucleic acid of the present invention encodes a p₂90 protein that comprises the amino acid sequence of SEQ ID NO: 4. In a particular embodiment of this type the nucleic acid comprises the nucleotide sequence of SEQ ID NO: 3. In another embodiment the nucleic acid comprises the nucleotide sequence of SEQ ID NO: 6.

The present invention also provides nucleic acids (e.g., DNA molecules) of 18 nucleotides or more that hybridize under stringent conditions with the nucleic acid having the nucleotide sequence of SEQ ID NO: 1. In a particular embodiment, the nucleic acid comprises 120 nucleotides or more and hybridizes under stringent conditions with the nucleic acid having the nucleotide sequence of SEQ ID NO: 1. In another embodiment, the nucleic acid comprises 300 nucleotides or more and hybridizes under stringent conditions with the nucleic acid having the nucleotide sequence of SEQ ID NO: 1. In still another embodiment, the nucleic acid comprises 900 nucleotides or more and hybridizes under stringent conditions with the nucleic acid having the nucleotide sequence of SEQ ID NO: 1. In yet another embodiment the nucleic acid comprises between 2000 to 3000 nucleotides and hybridizes under stringent conditions with the nucleic acid having the nucleotide sequence of SEQ ID NO: 1. In a related embodiment, the DNA molecule encodes a p₁90 protein and hybridizes under stringent conditions with the nucleic acid having the nucleotide sequence of SEQ ID NO: 1.

The present invention also provides nucleic acids (e.g., DNA molecules) of 18 nucleotides or more that hybridize under stringent conditions with the nucleic acid having the nucleotide sequence of SEQ ID NO: 3. In a particular embodiment, the nucleic acid comprises 120 nucleotides or more and hybridizes under stringent conditions with the nucleic acid having the nucleotide sequence of SEQ ID NO: 3. In another embodiment, the nucleic acid comprises 300 nucleotides or more and hybridizes under stringent conditions with the nucleic acid having the nucleotide sequence of SEQ ID NO: 3. In still another embodiment, the nucleic acid comprises 900 nucleotides or more and hybridizes under stringent conditions with the nucleic acid having the nucleotide sequence of SEQ ID NO: 3. In yet another embodiment the nucleic acid comprises between 1500 to 2600 nucleotides and hybridizes under stringent conditions with the nucleic acid having the nucleotide sequence of SEQ ID NO: 3. In a related embodiment, the DNA molecule encodes a p₂90 protein and hybridizes under stringent conditions with the nucleic acid having the nucleotide sequence of SEQ ID NO: 3.

The present invention also provides vectors that comprise one or more of the nucleic acids of the present invention. In one embodiment of this type, the vector is an expression vector. Preferably the nucleic acids of the present invention are operatively linked to a transcriptional control sequence in the expression vectors.

The expression vectors of the present invention can be used to express one or more p₁90 proteins, p₂90 proteins, antigenic fragments of the p₁90 proteins and/or p₂90 proteins, and/or corresponding chimeric polypeptides. In one such embodiment, the expression vector is a plasmid that can function in E. coli. In a particular embodiment, the expression vector is the EGT1 plasmid. In one such embodiment, the expression vector is an EGT1 plasmid that expresses p₁90 that has the amino acid sequence of SEQ ID NO: 2, and which has the BCCM accession No. LMBP 5690. In an alternative embodiment, the expression vector is an EGT1 plasmid that expresses p₂90 that has the amino acid sequence of SEQ ID NO: 4, and which has the BCCM accession No. LMBP 5691.

The present invention further provides host cells that comprise the vectors of the present invention. In a particular embodiment, the host cell expresses one or more p₁90 proteins, p₂90 proteins, antigenic fragments of the p₁90 proteins and/or p₂90 proteins, and/or corresponding chimeric polypeptides. In one embodiment the host cell comprises a plasmid that expresses p₁90 that comprises the amino acid sequence of SEQ ID NO: 2. In a particular embodiment of this type, the plasmid is an EGT1 plasmid that has the BCCM accession No. LMBP 5690. In another embodiment the host cell comprises a plasmid that expresses p₂90 that comprises the amino acid sequence of SEQ ID NO: 4. In a particular embodiment of this type, the plasmid is an EGT1 plasmid that has the BCCM accession No. LMBP 5691. Preferably, the host cell is an E. coli cell.

The present invention also provides methods for expressing and/or producing one or more p₁90 proteins, p₂90 proteins, antigenic fragments of the p₁90 proteins and/or p₂90 proteins, and/or corresponding chimeric polypeptides. One such embodiment is culturing a host cell of the present invention in a culture medium. In a particular embodiment, the method further comprises isolating the p₁90 protein(s), p₂90 protein(s), antigenic fragment(s) of the p₁90 protein(s) and/or p₂90 protein(s), and/or corresponding chimeric polypeptide(s). In one such embodiment, the host cell is an E. coli cell. In a particular embodiment the host cell comprises a EGT1 plasmid that expresses p₁90 that comprises the amino acid sequence of SEQ ID NO: 2. In a particular embodiment of this type, the plasmid is an EGT1 plasmid that has the BCCM accession No. LMBP 5690. In another embodiment the host cell comprises a EGT1 plasmid that expresses p₂90 that comprises the amino acid sequence of SEQ ID NO: 4. In a particular embodiment of this type, the plasmid is an EGT1 plasmid that has the BCCM accession No. LMBP 5691.

In another aspect of the present invention, immunogenic compositions are provided comprising the proteins, and/or antigenic fragments, and/or recombinant host cells, and/or bacterins of the present invention. In a preferred embodiment of this type, an immunogenic composition of the present invention is a vaccine. Accordingly, the vaccines of the present invention can comprise any of the immunogenic compositions of the present invention. Preferred vaccines protect fish against SRS, either alone or in multivalent vaccines that may also protect against other pathogens. In a related embodiment, a vaccine is a naked DNA vaccine that comprises a recombinant DNA vector that comprises an antigen of the present invention or an antigenic fragment thereof.

Immunogenic compositions of the present invention comprise antigenically effective amounts of a p₁90 protein of the present invention and/or of an antigenic fragment thereof; and/or an antigenically effective amount of a p₂90 protein of the present invention and/or of an antigenic fragment thereof; and/or antigenically effective amounts of a mixture of a p₁90 protein of the present invention and/or of an antigenic fragment thereof and/or a p₂90 protein of the present invention and/or of an antigenic fragment thereof.

In a particular embodiment, the immunogenic composition comprises a bacterin that comprises the plasmid having the BCCM accession No. LMBP 5690. In another embodiment the immunogenic composition comprises a bacterin that comprises the plasmid having the BCCM accession No. LMBP 5691. In still another embodiment, the immunogenic composition comprises both a bacterin that comprises the plasmid having the BCCM accession No. LMBP 5690 and a bacterin that comprises the plasmid having the BCCM accession No. LMBP 5691.

The vaccines of the present invention can further include an adjuvant. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, and dinitrophenol.

A vaccine and/or immunogenic composition of the present invention can further comprise one or more additional P. salmonis proteins or an antigenic fragment thereof. In one such embodiment the P. salmonis protein is the ^{Ps}45 protein. In a particular embodiment of this type, the ^{Ps}45 protein can comprise the amino acid sequence of SEQ ID NO: 8. In another embodiment, the vaccine and/or immunogenic composition can further comprise a bacterin comprised of a Yersinia ruckeri cell having the BCCM accession No. of LMG P-22044. In another embodiment, the bacterin is a Yersinia ruckeri cell BCCM accession No. LMG P-22511. In still another vaccine and/or immunogenic composition a bacterin comprising both the Yersinia ruckeri cell having the BCCM accession No. of LMG P-22044 and a Yersinia ruckeri cell BCCM accession No. LMG P-22511 is included.

In another embodiment, a vaccine of the present invention further comprises one or more antigens obtained from an Infectious Pancreatic Necrosis (IPN) virus. These recombinant proteins are preferably expressed by transformed yeast, Pichia pastoris. In one such embodiment, the antigen obtained from the IPN virus is the VP2 var protein or antigenic fragment thereof. In another embodiment the antigen obtained from the IPN virus is the VP3 protein or antigenic fragment thereof. In a preferred embodiment, the vaccine comprises both the VP2 var protein or antigenic fragment thereof and the VP3 protein or antigenic fragment thereof.

In one embodiment, an antigen is the portion of the VP2 var protein obtained from the transformed Pichia pastoris cell, BCCM Accession No. IHEM 20069. In another embodiment of this type, an antigen is the portion of the VP2 var protein obtained from the transformed Pichia pastoris cell, BCCM Accession No. IHEM 20070. In still another embodiment, an antigen is the portion of the VP3 protein obtained from the transformed Pichia pastoris cell, BCCM Accession No. IHEM 20071. In yet another embodiment, an antigen is the portion of the VP3 protein obtained from the transformed Pichia pastoris cell, BCCM Accession No. IHEM 20072. In a particular embodiment the vaccine comprises antigens from transformed Pichia pastoris cells, BCCM Accession No. IHEM 20069 and BCCM Accession No. IHEM 20071. In another embodiment the vaccine comprises antigens from transformed Pichia pastoris cells. BCCM Accession No. IHEM 20070, and BCCM Accession No. HEM 20072.

In still another embodiment a vaccine of the present invention comprises one or more antigens obtained from Aeromonas salmonicida. In a particular embodiment, the Aeromonas salmonicida comprising the antigens is prepared from a culture grown under iron-depleted conditions. In another embodiment, the Aeromonas salmonicida comprising the antigens is prepared from a culture grown under iron-supplemented conditions. In a preferred embodiment, two sets of Aeromonas salmonicida antigens are employed in the vaccine, one set from a culture grown under iron-depleted conditions the other set from a culture grown under iron-supplemented conditions. In a particular embodiment, a multivalent vaccine comprises one or more antigens from the present invention along with antigens from Piscirickettsia salmonis, IPN, and/or Aeromonas salmonicida.

The present invention also provides methods of protecting a fish from salmonid rickettsial septicemia (SRS), or SRS along with one or more other pathogenic disease(s) through the vaccination of the fish with a vaccine of the present invention. In a particular embodiment the other disease is Infectious Pancreatic Necrosis. In another embodiment the other disease is furunculosis. In still another embodiment the method of protecting the fish includes protecting against SRS, Infectious Pancreatic Necrosis, and furunculosis (caused by Aeromonas salmonicida).

The vaccines of the present invention can be administered by any method. In one embodiment a vaccine of the present invention is administered by immersion. In another embodiment a vaccine of the present invention is administered by injection. In yet another embodiment a vaccine of the present invention is administered by oral administration.

In addition, related booster vaccines are also provided by the present invention. The administration of a given booster vaccine is preferably performed through oral administration.

Any fish may be the recipient of the vaccines of the present invention. Examples of recipient fish are listed below. In a particular embodiment, the fish is a teleost. In a preferred embodiment, the telost is a salmonid. In a more preferred embodiment the salmonid is a salmon. In one such embodiment the salmon is a Salmo salar (Atlantic salmon). In another embodiment the salmon is an Oncorhynchus kisutch (coho salmon). In yet another embodiment the salmonid is an Oncorhynchus mykiss (rainbow trout).

Accordingly, it is a principal object of the present invention to provide a vaccine that protects salmonids against SRS.

It is a further object of the present invention to provide a vaccine that protects fish from salmonid rickettsial septicemia (SRS) and Infectious Pancreatic Necrosis (IPN).

It is a further object of the present invention to provide an effective way to protect against assorted fish infections by providing a multivalent vaccine. It is a further object of the present invention to provide a protocol that can lead to the successful vaccination of fish in captivity.

It is a further object of the present invention to provide a DNA construct that encodes the p₁90 protein or variant thereof.

It is a further object of the present invention to provide a DNA construct that encodes the p₂90 protein or variant thereof.

It is a further object of the present invention to provide a polypeptide having an amino acid sequence of SEQ ID NO: 2, or an antigenic fragment thereof.

It is a further object of the present invention to provide a polypeptide having an amino acid sequence of SEQ ID NO: 4, or an antigenic fragment thereof.

It is a further object of the present invention to provide a recombinant subunit vaccine against SRS.

It is a further object of the present invention to provide inactivated recombinant bacterial vectors encoding specific antigens to be used in vaccines against SRS.

These and other aspects of the present invention will be better appreciated by reference to the following drawings and Detailed Description.

### BRIEF DESCRIPTION OF THE DRAWINGS

FIG. 1 illustrates the cumulative percent mortality in different test groups of fish after vaccination. The curves show the results of the saline controls denoted by diamonds, the adjuvant controls denoted by squares, the 90 kDa ORF 1 [p₁90] vaccine denoted by triangles, and the 90 kDa ORF 2 [p₂90] vaccine denoted by “X”'s.

### DETAILED DESCRIPTION OF THE INVENTION

The present invention provides safe and effective vaccines to protect fish against Piscirickettsia salmonis infections. In addition, the present invention provides methods of vaccinating fish to protect them from Piscirickettsia salmonis and Piscirickettsia-like bacteria. Moreover, the present invention provides vaccines that can protect vaccinated fish from Piscirickettsia salmonis and other unrelated pathogens, such as the IPN virus. Methods of making the vaccines of the present invention are also provided. The vaccines of the present invention (including booster vaccines) can be administered to fish by a number of means including by immersion, by injection, and/or through oral administration.

Notably, US Published Patent Application No. US20070207165 (A1) and WO2006037383(A1) specifically teach the use a recombinant Yersinia ruckeri vector to express P. salmonis proteins, and indeed, exemplify the successful use of Yersinia ruckeri to express the ^{Ps}p45 protein. However, after considerable time and effort, Yersinia ruckeri proved to be an inappropriate host cell for expressing either p₁90 or p₂90 due both to instability, and the inability to demonstrate expression of these proteins. Surprisingly, and contrary to the earlier teachings of US20070207165 (A1) and WO2006037383(A1), E. coli cells proved to be the preferred recombinant host cell to express either p₁90 or p₂90.

Accordingly, in a particular aspect of the present invention the p₁90 and p₂90 proteins are expressed in recombinant E. coli host cells containing pEGT1 plasmids. Two corresponding recombinant E. coli HMS174(DE3)/pEGT1 plasmids encoding these proteins were deposited with the:

Belgian Coordinated Collections of Microorganisms (BCCM) under the terms of the Budapest Treaty and represented by:

BCCM/LMBP

Department of Molecular Biology

Ghent University

Fiers-Schell-Van Mantagu Building

Technologiepark 927

B-9052 Zwijnaarde

### The International Depository Authority:

Belgian Coordinated Collections of Microorganisms (BCCM™)

Laboratorium voor Moleculaire Biologie-Plasmidencollectie (LMBP)

Universiteit Gent

Technologiepark 927

B-9052 Gent-Zwijnaarde, Belgium

Both plasmid deposits were all made on Oct. 19, 2007.
p₁90
   E. coli HMS174(DE3)/pEGT1/AL-ORF1-90 kDa
   BCCM accession No. LMBP 5690
p₂90
   E. coli HMS174(DE3)/pEGT1/AL-ORF2-90 kDa
   BCCM accession No. LMBP 5691

The present invention also provides vaccines against SRS that further comprise and/or encode one or more additional P. salmonis antigens. Such additional antigens include those described by US Published Patent Application No. US20070207165 (A1) and those described by WO2006037383(A1), the contents of both of which are hereby incorporated by reference in their entireties. These antigens include isolated P. salmonis ^{Ps}p45 protein comprising the amino acid sequence of SEQ ID NO: 7 (complete ^{Ps}p45 protein) or SEQ ID NO: 8 (^{Ps}p45 protein without the signal sequence) and antigenic fragments thereof.

Additional antigens described by US20070207165 (A1) and WO2006037383(A1), see also Table 14 below, include those comprising the amino acid sequence of SEQ ID NO: 9 (which shows homology with a protein family coding for AMP-binding enzymes), SEQ ID NO: 10 (which shows no homology to any protein family), SEQ ID NO: 11 (which shows no homology to any protein family), SEQ ID NO: 12 (which shows homology to the DDE endonuclease family and in particular to the integrase core domain), SEQ ID NO: 13 (which shows homology to transposases), SEQ ID NO: 14 (which shows some homology to the HlyD family of secretory proteins), and/or SEQ ID NO: 15 (which shows homology to the intergral membrane AcrB/AcrD/AcrB protein family). These antigens can be expressed e.g., by nucleic acids that encode one to or more of these amino acid sequences.

As indicated above, ^{Ps}p45 protein can be produced by recombinant Yersinia ruckeri cells deposited with the Belgian Coordinated Collections of Microorganisms (“BCCM”) located at:

BCCM Laboratorium voor Microbiologie—Bacteriënverzameling (LMG) Universiteit Gent, K. L. Ledeganckstraat 35, B-9000 Gent, Belgium
Strain Name: Yersinia ruckeri 224/pGEM5ZF+/45 kDa/S
   BCCM accession No. LMG P-22044, deposited on Sep. 11, 2003.
Strain Name: Yersinia ruckeri 224/pGEM5ZF+/75 kDa
   BCCM accession No. LMG P-22511, deposited on May 27, 2004.

The present invention also provides combination vaccines against SRS and IPN(SRS/IPN vaccines) that comprise one or more inventive P. salmonis 90 kDa antigens (e.g., p₁90 and/or p₂90) optionally in combination with any of the ^{Ps}p45 proteins or SRS antigens noted above, in combination with one or more antigens obtained from an Infectious Pancreatic Necrosis (IPN) virus. These recombinant proteins (IPN antigens) are preferably expressed by transformed yeast, Pichia pastoris.

In one such embodiment, the antigen obtained from the IPN virus is the VP2 var protein or an antigenic fragment thereof. In a particular embodiment, the antigen is the VP2 var protein obtained from the transformed Pichia pastoris cell, BCCM Accession No. IHEM 20069 and/or from the transformed Pichia pastoris cell, BCCM Accession No. IHEM 20070. In another embodiment, the antigen obtained from the IPN virus is the VP3 protein or an antigenic fragment thereof. In a particular embodiment of this type, the antigen is the VP3 protein obtained from the transformed Pichia pastoris cell, BCCM Accession No. IHEM 20071 and/or from the transformed Pichia pastoris cell BCCM Accession No. IHEM 20072. In one embodiment of the present invention, the inventive SRS/IPN vaccine comprises at least one VP2var antigen and one VP3 antigen.

Four recombinant Pichia pastoris yeast cells were deposited with the to following depository:

BCCM

Institut Scientifique de la Santé Publique—Louis Pasteur (IHEM)

Section mycologie

J. Wytsmanstraat 14 Rue J. Wytsman

B-1050 Brussels, Belgium

These deposits were all made on Sep. 11, 2003.

Strain name: Pichia pastoris GS115/pPICZaB/VP2var/MUT+46
BCCM Accession No. IHEM 20069

Strain name: Pichia pastoris SMD1168/pPICZaB/VP2 367.5
BCCM Accession No. IHEM 20070

Strain name: Pichia pastoris KM71/pPICZaB/VP3/MUTs 30:11
BCCM Accession No. IHEM 20071

Strain name: Pichia pastoris GS115/pPICZaB/VP3 112.15
BCCM Accession No. IHEM 20072

As used herein the following terms shall have the definitions set out below:

As used herein the term “p₁90” is used interchangably with the term “ORF1” and denotes a specific protein that is about 90 kDa in molecular weight. In a particular embodiment, p₁90 comprises the amino acid sequence of SEQ ID NO: 2, which is encoded by the nucleotide sequence SEQ ID NO: 1 and the nucleotide sequence SEQ ID NO: 5, which was optimized for E. coli codon usage. p₁90 is encoded by an EGT1 plasmid deposited with the BCCM™/LMBP Collection having ascension number LMP 5690.

As used herein the term “p₂90” is used interchangably with the term “ORF2” and denotes a specific protein that is about 90 kDa in molecular weight. In a particular embodiment, p₂ 90 comprises the amino acid sequence of SEQ ID NO: 4, which is encoded by the nucleotide sequence SEQ ID NO: 3 and the nucleotide sequence SEQ ID NO: 6, which was optimized for E. coli codon usage. p₂90 is encoded by an EGT1 plasmid deposited with the BCCM™/LMBP Collection having ascension number LMP 5691.

As used herein the term “polypeptide” is used interchangeably with the term “protein” and is further meant to encompass peptides. Therefore, as used to herein, a polypeptide is a polymer of two or more amino acids joined together by peptide linkages. Preferably, a polypeptide is a polymer comprising twenty or more amino acid residues joined together by peptide linkages, whereas a peptide comprises two to twenty amino acid residues joined together by peptide linkages.

As used herein a polypeptide “consisting essentially of” or that “consists essentially of” a specified amino acid sequence is a polypeptide that (i) retains an important characteristic of the polypeptide comprising that amino acid sequence, e.g., the antigenicity of at least one epitope of the inventive 90 kDa protein(s), and (ii) further comprises the identical amino acid sequence(s), except it consists of plus or minus 10% (or a lower percentage), and preferably plus or minus 5% (or a lower percentage) of the amino acid residues. In a particular embodiment, additional amino acid residues included as part of the polypeptide are part of a linked Tag, such as a C-terminal His₆ Tag.

A molecule is “antigenic” when it is capable of specifically interacting with an antigen recognition molecule of the immune system, such as an immunoglobulin (antibody) or T cell antigen receptor. An antigenic polypeptide (and/or fragment of the polypeptide) contains at least 6, and preferably at least 12 or more amino acid residues. An antigenic portion of a molecule can be that portion that is immunodominant for recognition by an antibody or a T cell receptor, and/or it can be a portion used to generate an antibody to the molecule by conjugating an immunogenic portion of the antigen to a carrier molecule for immunization. A molecule that is antigenic need not be itself immunogenic, i.e., capable of eliciting an immune response without a carrier.

As used herein the term “antigenic fragment” of a particular protein is a fragment of that protein that is antigenic. For example, an antigenic fragment of a p₁90 protein or a p₂90 protein can be any antigenic fragment of the p₁90 protein or p₂90 protein respectively, including large fragments that are missing as little as a single amino acid from the full-length protein. In a particular embodiment, an antigenic fragment of the p₁90 protein or a p₂90 protein contains between 12 and 800 amino acid residues. In another embodiment, an antigenic fragment of the p₁90 protein or a p₂90 protein contains between 25 and 250 amino acid residues. In yet another embodiment, an antigenic fragment of a p₁90 protein or a p₂90 protein contains 100 amino acid residues or more, but fewer than 600 amino acid residues. In still another embodiment, an antigenic fragment of a p₁90 protein or a p₂90 protein contains 250 amino acid residues or more, but fewer than 600 amino acid residues. In yet another embodiment, an antigenic fragment of a p₁90 protein or a p₂90 protein contains 400 amino acid residues or more, but fewer than 600 amino acid residues.

An antigenic fragment of a given p₁90 protein or a p₂90 protein can be obtained from a recombinant source, from a protein isolated from natural sources, or through chemical synthesis. Similarly, an antigenic fragment can be obtained following the proteolytic digestion of such p₁90 proteins, p₂90 proteins or fragments of either. Alternatively, an antigenic fragment of the present invention can be generated by recombinant expression, or alternatively, through peptide synthesis.

As used herein, a multivalent vaccine is a vaccine that comprises two or more different antigens. In a particular embodiment of this type, the multivalent vaccine stimulates the immune system of the recipient against two or more different pathogens. Specific multivalent vaccines are exemplified below.

As used herein the term “chimeric protein” is used interchangeably with the terms “chimeric polypeptide” and “chimeric peptide” and is meant to include fusion proteins, polypeptides, and peptides. A “chimeric protein” comprising a p₁90 and/or p₂90 protein of the present invention comprises at least a portion of a particular protein (e.g., p₁90) joined via a peptide bond to at least a portion of a different protein (e.g., a non-p₁90 protein). A chimeric protein of the present invention also can comprise two or more different proteins and/or portions thereof, including a chimeric p₁90-p₂90 protein. Chimeric proteins of the present invention also can have additional structural, regulatory, and/or catalytic properties. As used herein a chimeric protein can contain multiple additions to at least a portion of a given protein, e.g., a chimeric protein can comprise both a His₆Tag and an alternative signal sequence. In a particular embodiment, a non-p₁90 (or non-p₂90) portion of the chimeric protein functions as a means of detecting and/or isolating the chimeric protein or fragment thereof after a recombinant nucleotide encoding the given protein or antigenic fragment thereof is expressed. Non-p₁90 (or non-p₂90) protein amino acid sequences are generally, but not always, either amino- or carboxy-terminal to the protein sequence.

As used herein one amino acid sequence is 100% “identical” to a second amino acid sequence when the amino acid residues of both sequences are identical. Accordingly, an amino acid sequence is 50% “identical” to a second is amino acid sequence when 50% of the amino acid residues of the two amino acid sequences are identical. The sequence comparison is performed over a contiguous block of amino acid residues comprised by a given protein, e.g., a protein, or a portion of the polypeptide being compared. In a particular embodiment, selected deletions or insertions that could otherwise alter the correspondence between the two amino acid sequences are taken into account.

As used herein, DNA and protein sequence percent identity can be determined using C, MacVector (MacVector, Inc. Cary, N.C. 27519), Vector NTI (Informax, Inc. MD), Oxford Molecular Group PLC (1996) and the Clustal W algorithm with the alignment default parameters, and default parameters for identity. These commercially available programs can also be used to determine sequence similarity using the same or analogous default parameters. Alternatively, an Advanced Blast search under the default filter conditions can be used, e.g., using the GCG (Genetics Computer Group, Program Manual for the GCG Package, Version 7, Madison, Wis.) pileup program using the default parameters.

As used herein a “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. When referring to a nucleic acid that is double stranded both the “sense” strand and the complementary “antisense” strand are intended to be included. Thus a nucleic acid that is hybridizable to SEQ ID NOs: 1 or 3, for example, can be either hybridizable to the “sense” strand of the respective sequence, or to the “antisense” strand which can be readily determined from the respective sense strands listed in the Sequence Listing provided herein. The individual components of a nucleic acid are referred to as nucleotides.

A DNA “coding sequence” is a double-stranded DNA sequence that is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A nucleotide coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which can then be trans-RNA spliced, if, when, and where appropriate, and translated into the protein encoded by the coding sequence.

A nucleotide sequence is “operatively linked” to an expression control sequence when the expression control sequence controls or regulates the transcription and translation of that nucleotide sequence. The term operatively linked includes having an appropriate start signal.

A “heterologous nucleotide sequence” as used herein is a nucleotide sequence that is added by recombinant methods to a nucleotide sequence is encoding a polypeptide of the present invention or encoding a fragment thereof. (i.e., an antigenic fragment), to form a nucleic acid that is not naturally formed in nature. Such nucleic acids can e.g., encode chimeric proteins. In addition, as used herein, a heterologous nucleotide sequence need not be a single contiguous nucleotide sequence, but can include multiple non-contiguous nucleotide sequences that have been combined with a nucleotide sequence encoding a polypeptide of the present invention, or a portion thereof. A heterologous nucleotide sequence can comprise non-coding sequences including restriction sites, regulatory sites, promoters and the like. In still another embodiment the heterologous nucleotide can function as a means of detecting a nucleic acid of the present invention.

The present invention provides heterologous nucleotide sequences that when combined with nucleotide sequences encoding a polypeptide of the invention or a fragment thereof, are necessary and sufficient to encode all of the chimeric proteins of the present invention. In a particular embodiment, the polypeptide comprises the amino acid sequence of SEQ ID NO: 2.

As used herein, a bacterium is said to be “recombinant” when the nucleotide sequence of the DNA that it naturally contains has been purposely altered by at least one nucleotide addition, deletion, and/or modification through genetic engineering. A recombinant bacterin is an inactivated or killed recombinant bacterium.

The phrase “binding to” or “binds to” in regard to a ligand binding to a polypeptide (e.g., antigen to an antibody) is used herein to include any or all such specific interactions that lead to a protein-ligand binding complex. This can include processes such as covalent, ionic (electrostatic and/or charged), hydrophobic and hydrogen bonding, but does not include non-specific associations such as solvent preferences.

As used herein a “small organic molecule” is an organic compound [or organic compound complexed with an inorganic compound (e.g., metal)] that has a molecular weight of less than 3 kDa.

As used herein the terms “approximately” and “about” are used to signify that a value is within twenty percent of the indicated value i.e., an amino acid is sequence containing “approximately” 400 amino acid residues can contain between 320 and 480 amino acid residues.

As used herein the unit “^{o} days” denotes the number of days of incubation following the vaccination of a fish, multiplied by the average temperature in ° C. for that incubation.

### Nucleic Acids Encoding the Polypeptides of the Present Invention

A nucleic acid, such as a cDNA, that encodes a polypeptide of the present invention, can be placed into a vector, e.g., a recombinant bacterial host cell, to express a protein and/or antigen of the present invention, e.g., the p₁ 90 and p₂ 90 proteins. Such recombinant host cells can be inactivated, e.g., disrupted and converted to bacterins, and used in immunogenic compositions such as vaccines.

In addition, obtaining and/or constructing a DNA that encodes one of the polypeptides of the present invention, including those encoding p₁ 90 and/or p₂ 90, or antigenic fragments thereof, facilitates the production of economically important quantities of the protein or antigenic fragments thereof. The large quantities of the proteins and/or antigenic fragments thereof produced are useful for making certain vaccines of the present invention.

Accordingly, the present invention provides specific nucleotide constructs that allow for the expression and isolation of large quantities of the proteins and/or antigens of the present invention, such as the p₁ 90 and/or p₂90 proteins. These nucleic acids can further contain heterologous nucleotide sequences. To express a recombinant protein of the present invention in a host cell, an expression vector can be constructed comprising the corresponding cDNA. The present invention therefore, provides expression vectors containing nucleic acids encoding the proteins of the present invention, including variants thereof.

Due to the degeneracy of nucleotide coding sequences, other DNA sequences which encode substantially the same amino acid sequence as a nucleic acid encoding a polypeptide of the present invention may be used in the practice of the present invention. These include, but are not limited to, allelic genes, homologous genes from other strains, and/or those that are altered by the substitution of different codons that encode the same amino acid residue within the sequence, thus producing a silent change. Host cells comprising the expression vectors of the present invention are also provided. One particular host cell is an E. coli cell.

General methods for the cloning of cDNAs and expression of their corresponding recombinant proteins have been described [see Sambrook and Russell, Molecular Cloning, A laboratory Manual, 3^{rd} edition, Cold Spring Harbor Laboratory Press, Cold Spring Harbor L.I. (2000)]. The particular methodology used herein is described in the Examples below. Preferably, all of the nucleic acid constructs of the present invention are sequence confirmed.

In addition, any technique for mutagenesis known in the art can be used to modify a native p₁ 90 or p₂ 90 protein of the present invention, including but not limited to, in vitro site-directed mutagenesis [Hutchinson et al., J. Biol. Chem., 253:6551 (1978); Zoller and Smith, DNA, 3:479-488 (1984); Oliphant et al., Gene, 44:177 (1986); Hutchinson et al., Proc. Natl. Acad. Sci. U.S.A., 83:710 (1986); Wang and Malcolm, Bio Techniques 26:680-682 (1999) the contents of which are hereby incorporated by reference in their entireties]. The use of TAB@ linkers (Pharmacia), etc. and PCR techniques also can be employed for site directed mutagenesis [see Higuchi, “Using PCR to Engineer DNA”, in PCR Technology: Principles and Applications for DNA Amplification, H. Erlich, ed., Stockton Press, Chapter 6, pp. 61-70 (1989)].

The present invention also provides nucleic acids that hybridize to nucleic acids comprising the nucleotide sequences of the present invention. A nucleic acid is “hybridizable” to another nucleic acid, such as a cDNA, genomic DNA, or RNA, when a single stranded form of the nucleic acid can anneal to the other nucleic acid under the appropriate conditions of temperature and solution ionic strength [see Sambrook and Russell, Molecular Cloning, A laboratory Manual, 3^{rd} edition, Cold Spring Harbor Laboratory Press, Cold Spring Harbor L.I. (2000)].

The conditions of temperature and ionic strength determine the “stringency” of the hybridization. For preliminary screening for homologous nucleotides, low stringency hybridization conditions, corresponding to a Tₘ of 55° C., can be used, e.g., 5× saline sodium citrate (SSC), 0.1% sodium dodecyl sufate (SDS), 0.25% milk, and no formamide; or 30% formamide, 5×SSC, 0.5% SDS. Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleotides having those sequences. The relative stability (corresponding to higher Tₘ) of nucleotide hybridizations decreases in the following order: RNA:RNA, DNA:RNA, DNA:DNA. For hybrids of greater than 100 nucleotides in length, equations for calculating Tₘ have been derived [see Sambrook and Russell, Molecular Cloning, A laboratory Manual, 3^{rd} edition, Cold Spring Harbor Laboratory Press, Cold Spring Harbor L.I. (2000)]. For hybridization with shorter nucleic acids, i.e., oligonucleotides, the position of mismatches becomes more important, and the length of the oligonucleotide determines its specificity.

Depending upon circumstances a suitable minimal length for a hybridizable nucleic acid can be at least about 12 nucleotides; or at least about 18 nucleotides; or the length can be at least about 24 nucleotides; or at least about 36 nucleotides. Alternatively, the minimum length can be at least about 48 or at least about 72 nucleotides, or longer, as indicated above. In a specific embodiment, the term “standard hybridization conditions” refers to a Tₘ of 55° C., and utilizes conditions as set forth above. Under more stringent conditions, the Tₘ is 60° C., to and under even more stringent conditions, the Tₘ is 65° C. for both hybridization and wash conditions, respectively.

### Polypeptides of the Present Invention

The present invention provides isolated and/or recombinant polypeptides, is including all of the antigens of the present invention, e.g., the p₁ 90 and/or p₂ 90 proteins (plus or minus an amino-terminal signal peptide), variants thereof, antigenic fragments thereof, and chimeric proteins thereof. In addition, polypeptides containing altered sequences in which functionally equivalent amino acid residues are substituted for those within the wild type amino acid sequence resulting in a conservative amino acid substitution, are also provided by the present invention.

For example, one or more of these amino acid residues within the sequence can be substituted by another amino acid of a similar polarity, which acts as a functional equivalent, resulting in a silent alteration. Substitutes for an amino acid within the sequence may be selected from other members of the class to which the amino acid belongs.

For example, the nonpolar amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine and lysine. The negatively charged (acidic) amino acids include aspartic acid and glutamic acid.

Particularly preferred conserved amino acid exchanges are:
(a) Lys for Arg or vice versa such that a positive charge may be maintained;
(b) Glu for Asp or vice versa such that a negative charge may be maintained;
(c) Ser for Thr or vice versa such that a free —OH can be maintained;
(d) Gln for Asn or vice versa such that a free NH₂ can be maintained; and
(e) Ile for Leu or for Val or vice versa as being roughly equivalent hydrophobic amino acids.

All of the polypeptides of the present invention, including antigenic fragments, also can be part of a chimeric protein. In a specific embodiment, a chimeric polypeptide is expressed in a prokaryotic cell. Such a chimeric protein can be a fusion protein used to isolate a polypeptide of the present invention, is through the use of an affinity column that is specific for a protein fused to the p₁ 90 and/or p₂ 90 proteins, for example. Examples of such fusion proteins include: a glutathione-S-transferase (GST) fusion protein, a maltose-binding protein (MBP) fusion protein, a FLAG-tagged fusion protein, or a poly-histidine-tagged fusion protein. Specific linker sequences such as a Ser-Gly linker can also be part of such a fusion protein.

Indeed, the expression of one or more of the inventive proteins, as a fusion protein, can facilitate stable expression, and/or allow for purification based on the properties of the fusion partner. Thus the purification of the recombinant polypeptides of the present invention can be simplified through the use of fusion proteins having affinity Tags. For example, GST binds glutathione conjugated to a solid support matrix, MBP binds to a maltose matrix, and poly-histidine chelates to a Ni-chelation support matrix [see Hochuli et al., Biotechnology 6:1321-1325 (1998)].

The fusion protein can be eluted from the specific matrix with appropriate buffers, or by treating with a protease that is specific for a cleavage site that has been genetically engineered in between a p₁ 90 and/or p₂ 90 protein, for example, and its fusion partner. Alternatively, a p₁ 90 and/or p₂ 90 protein can be combined with a marker protein such as green fluorescent protein [Waldo et al., Nature Biotech. 17:691-695 (1999); U.S. Pat. No. 5,625,048 and WO 97/26333, the contents of which are hereby incorporated by reference in their entireties].

Alternatively or in addition, other column chromatography steps (e.g., gel filtration, ion exchange, affinity chromatography etc.) can be used to purify the recombinant polypeptides of the present invention (see below). In many cases, such column chromatography steps employ high performance liquid chromatography or analogous methods in place of the more classical gravity-based procedures.

In addition, the polypeptides of the present invention, including the p₁ 90 and/or p₂ 90 proteins, and antigenic fragments thereof, can be chemically synthesized [see e.g., Synthetic Peptides: A User's Guide, W.H.Freeman & Co., New York, N.Y., pp. 382, Grant, ed. (1992)].

### General Polypeptide Purification Procedures

Generally, initial steps for purifying a polypeptide of the present invention can include salting in or salting out, in ammonium sulfate fractionations; solvent exclusion fractionations, e.g., an ethanol precipitation; detergent extractions to free membrane bound polypeptides, using such detergents as TRITON X-100, TWEEN-20 etc.; or high salt extractions. Solubilization of membrane proteins may also be achieved using aprotic solvents such as dimethyl sulfoxide and hexamethylphosphoramide. In addition, high speed ultracentrifugation may be used either alone or in conjunction with other extraction techniques.

Generally good secondary isolation or purification steps include solid phase absorption using calcium phosphate gel, hydroxyapatite, or solid phase binding. Solid phase binding may be performed through ionic bonding, with either an anion exchanger, such as diethylaminoethyl (DEAE), or diethyl [2-hydroxypropyl]aminoethyl (QAE) SEPHADEX or cellulose; or with a cation exchanger such as carboxymethyl (CM) or sulfopropyl (SP) SEPHADEX or cellulose. Alternative means of solid phase binding includes the exploitation of hydrophobic interactions e.g., the use of a solid support such as phenylSepharose and a high salt buffer; affinity-binding immuno-binding, using e.g., a inventive protein bound to a suitable anti-p₁ 90 and/or anti-p₂ 90 selective antibody, respectfully, bound to an activated support. Other solid phase supports include those that contain specific dyes or lectins etc.

A further solid phase support technique that is often used at the end of the purification procedure relies on size exclusion, such as SEPHADEX and SEPHAROSE gels. Alternatively, a pressurized or centrifugal membrane technique, using size exclusion membrane filters may be employed. Oftentimes, these two methodologies are used in tandem.

Solid phase support separations are generally performed batch-wise with low-speed centrifugation, or by column chromatography. High performance liquid chromatography (HPLC), including such related techniques as FPLC, is presently the most common means of performing liquid chromatography. Size exclusion techniques may also be accomplished with the aid of low speed centrifugation. In addition size permeation techniques such as gel electrophoretic techniques may be employed. These techniques are generally performed in tubes, slabs or by capillary electrophoresis.

Almost all steps involving polypeptide purification employ a buffered solution. Unless otherwise specified, generally 25-100 mM concentrations of buffer salts are used. Low concentration buffers generally imply 5-25 mM concentrations. High concentration buffers generally imply concentrations of the buffering agent of between 0.1-2.0 M concentrations. Typical buffers can be purchased from most biochemical catalogues and include the classical buffers such as Tris, pyrophosphate, monophosphate and diphosphate and the Good buffers such as Mes, Hepes, Mops, Tricine and Ches [Good et al., Biochemistry, 5:467 (1966); Good and Izawa, Meth. Enzymol., 24B:53 (1972); and Fergunson and Good, Anal. Biochem., 104:300 (1980].

Materials to perform all of these techniques are available from a variety of commercial sources such as Sigma Chemical Company in St. Louis, Mo.

### Antibodies to the Polypeptides of the Present Invention

The polypeptides of the present invention, and antigenic fragments thereof, as produced by a recombinant source, or through chemical synthesis, or as isolated from natural sources; and variants, derivatives or analogs thereof, including fusion proteins, may be used as an immunogen to generate antibodies. Such antibodies include but are not limited to polyclonal, monoclonal, chimeric including single chain, Fab fragments, and a Fab expression library. Such antibodies can be used in diagnostic kits or as components in vaccines.

Specific anti-p₁ 90 and/or p₂ 90 protein antibodies of the invention, for example, may be cross-reactive, that is, they may recognize one specific 90 kDa protein, e.g., p₁ 90, or a closely related protein obtained from a different source (e.g., a Piscirickettsia-like bacterium). Polyclonal antibodies have greater likelihood of cross-reactivity. Alternatively, an antibody of the invention may be specific for a single form of an inventive protein, for example, such as a specific fragment of p₁ 90 that has the amino acid sequence of SEQ ID NO: 2, or a closely related variant thereof.

In a particular aspect of the present invention compositions and uses of is antibodies that are immunoreactive with a p₁ 90 and/or p₂ 90 protein are provided. Such antibodies “bind specifically” to the particular p₁ 90 and/or p₂ 90 protein respectively, meaning that they bind via antigen-binding sites of the antibody as compared to non-specific binding interactions.

The terms “antibody” and “antibodies” are used herein in their broadest sense, and include, without limitation, intact monoclonal and polyclonal antibodies as well as fragments such as Fv, Fab, and F(ab′) fragments, single-chain antibodies such as scFv, and various chain combinations. The antibodies may be prepared using a variety of well-known methods including, without limitation, immunization of animals having native or transgenic immune repertoires, phage display, hybridoma and recombinant cell culture.

Both polyclonal and monoclonal antibodies may be prepared by conventional techniques. [See, for example, Monoclonal Antibodies, Hybridomas: A New Dimension in Biological Analyses, Kennet et al. (eds.), Plenum Press, New York 37 (1980); and Antibodies: A Laboratory Manual, Harlow and Land (eds.), Cold Spring Harbor Laboratory Press, Cold Spring Harbor, N.Y., (1988)].

Various procedures known in the art may be used for the production of polyclonal antibodies to a particular p₁ 90 and/or p₂ 90 protein, variants or derivatives or analogs thereof. For the production of an antibody, various host animals can be immunized by injection with the p₁ 90 and/or p₂ 90 protein, variant or a derivative (e.g., or fusion protein) thereof or fragment thereof, including but not limited to rabbits, mice, rats, sheep, goats, etc. In one embodiment, the inventive protein can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, and dinitrophenol.

For preparation of monoclonal antibodies directed toward a given inventive protein, variant, or analog, or derivative thereof, any technique that provides for the production of antibody molecules by continuous cell lines in culture may be used. These include but are not limited to the hybridoma technique originally developed by Kohler and Milstein [Nature, 256:495-497 (1975)], as well as the trioma technique, and the human B cell hybridoma technique [Kozbor et al., Immunology Today, 4:72 (1983); Cote et al., Proc. Natl. Acad. Sci. U.S.A., 80:2026-2030 (1983)].

The monoclonal antibodies of the present invention include chimeric antibodies versions of antibodies originally produced in mice or other non-human animals. Techniques developed for the production of “chimeric antibodies” by splicing the genes from a mouse antibody molecule specific for a given inventive protein, for example, together with genes from a fish antibody of appropriate biological activity (e.g., a salmon) can be used. Such chimeric antibodies are within the scope of this invention [see in general, Morrison et al., J Bacteriol, 159:870 (1984); Neuberger et al., Nature, 312:604-608 (1984); Takeda et al., Nature, 314:452-454 (1985)].

Hybridoma cell lines that produce monoclonal antibodies specific for the polypeptides of the present invention are also provided by the present invention. Such hybridomas may be produced and identified by conventional techniques.

One method for producing such a hybridoma cell line comprises immunizing an animal with a polypeptide, harvesting spleen cells from the immunized animal, fusing the spleen cells to a myeloma cell line, thereby generating hybridoma cells, and identifying a hybridoma cell line that produces a monoclonal antibody that binds the polypeptide. The monoclonal antibodies produced by hybridomas may be recovered by conventional techniques.

According to the invention, techniques described for the production of single chain antibodies [U.S. Pat. Nos. 5,476,786, 5,132,405, and 4,946,778, the contents of which are hereby incorporated by reference in their entireties] can be adapted to produce p. salmonis protein-specific single chain antibodies, e.g., p₂ 90 protein-specific single chain antibodies. An additional embodiment of the invention utilizes the techniques described for the construction of Fab expression libraries [Huse et al., Science, 246:1275-1281 (1989)] to allow rapid and easy identification of monoclonal Fab fragments with the desired specificity for p₁ 90 and/or p₂ 90 protein, variant, derivative, and/or analog.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by such techniques as radioimmunoassay, enzyme-linked immunosorbant assay (ELISA), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays.

In one embodiment, antibody binding is detected by detecting a label, e.g., a fluorescent label such as fluorescene isothiocyanate (FITC), on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of a particular inventive protein, one may assay the hybridomas generated for a product which binds to a protein fragment containing such an epitope and choose those which do not cross-react with a modified inventive protein that does not contain that epitope. One can select an antibody specific to p₁ 90 and/or p₂ 90 from a particular source based on the positive specific binding with that specific protein.

### SRS Vaccines

The present invention provides SRS vaccines. One particular embodiment is a non-mineral oil injection prime vaccine comprising one or more antigens, as disclosed below. In one embodiment of this type, inactivated recombinant bacteria (bacterins) comprise one or more of the antigens of the present invention. The present invention also provides SRS vaccines that are designed to protect against one or more other fish pathogens. For example, furunculosis is an infectious ulcerative disease of salmon and trout caused by the bacterium Aeromonas salmonicida. In a particular embodiment, the vaccine will comprise in addition: Piscirickettsia salmonis component(s), two Infectious pancreatic necrosis (IPN) antigens as discussed herein, A. salmonicida as discussed below, Vibrio ordalii, Infectious Salmon Anemia, and/or Salmon Pancreatic Disease.

Other fish pathogens include, but are not limited to:

| PATHOGEN (antigen) | RELATED DISEASE |
|---|---|
| IPN virus | Infectious pancreatic necrosis |
| Vibrio anguillarum or | Vibrosis |
| Vibrio ordalii | |
| Vibrio salmonicida | Cold water Vibriosis (Hitra |
| | disease) |
| Moritella viscosus | Winter sores disease |
| Photobacterium damsela | Pasteurellosis |
| (subspecies Piscicida) | |
| Lactococcus garviae | Streptococcosis |
| Streptococcus iniae | |
| Moritella viscosus | Winter Sores |
| Noccardia kampachi | |
| Renibacterium salmoninarum | |
| ISA Virus | Infectious Salmon Anemia |
| IHN Virus | Infectious Heamorhagic Necrosis |
| SPD Virus | Salmon pancreatic disease |
| SD Virus | Sleeping disease |

The vaccines for these various diseases can be prepared from whole cells, bacterins, killed and/or attenuated virus, protein extracts, recombinant DNA vaccine vectors, isolated antigens, recombinant antigens and mixtures thereof. Under particular circumstances, as for Photobacterium damsela and Aeromonas salmonicida, the vaccines are preferably prepared from two separate cultures grown under iron-depleted conditions and iron-supplemented conditions, respectively.

In a particular embodiment, a vaccine comprises the p₁90 and/or p₂ 90 protein(s). In another embodiment, a vaccine comprises the p₁90 and/or p₂90 protein(s) in combination with one or more of the above-noted antigens from Piscirickettsia salmonis (e.g., the ^{Ps}45 protein). In another embodiment, a vaccine comprises the p₁90 and/or p₂90 protein(s), optionally in combination with IPN proteins, and optionally further in combination with one or more antigens from Piscirickettsia salmonis. In still another embodiment, the vaccine comprises the p₁ 90 and/or p₂ 90 protein(s) and/or one or more antigens from Piscirickettsia salmonis, one or more IPN proteins, and one or more antigens to control Aeromonas salmonicida. In a particular embodiment of this type, Aeromonas salmonicida antigens are two types of whole bacteria grown on bacterial growth media and killed by the addition of formalin.

For an SRS vaccine according to the invention, Escherichia coli was selected as the best candidate for hosting and expressing the p₁ 90 and/or p₂90 protein(s) of the present invention.

Two IPN viral antigens are exemplified below (see also WO 02/38770, the contents of which are hereby incorporated in its entireties). One of which is derived from Vp2, which is the major outer capsid protein and the other from Vp3, which is an internal protein of the IPN virus. The molecular weight of the Vp2 protein is 52 kDa, whereas that of the Vp3 protein is 30 kDa. The IPN proteins of the vaccines of the present invention are preferably purified recombinant proteins. In the Example 6 below, the IPN proteins are expressed and excreted by transformed yeast (Pichia pastoris) and then optionally purified from these yeast cells.

Antigens for a vaccine that also protects against furunculosis can be obtained from whole killed bacteria Aeromonas salmonicida (e.g., formalin-killed). Early A. salmonicida vaccines contain whole A. salmonicida bacteria grown in normal growth medium and then inactivated by the addition of formalin. These bacterins contain a mixture of antigens including the surface A-layer, inactivated proteases and lipopoly-saccharide. On the other hand when A salmonicida are grown in normal medium in the total absence of iron, a group of new antigens are expressed. These new antigens are termed iron-regulated outer membrane proteins (IROMPs). IROMPS are highly immunogenic and they provide enhanced protection relative to vaccines containing inactivated A. salmonicida grown in normal medium. Four IROMP proteins having molecular weights of 82 kDa, 77 kDa, 72 kDa and 70 kDa respectively have been identified.

The primary and secondary antibody responses to IROMP antigens in Atlantic salmon (Salmo salar) immunized with A+ (iron plus) and A− (iron minus) Aeromonas salmonicida bacterins have been reported [O'Dowd et al., Fish & Shellfish Immunology 9:125-138 (1999)]. Thus particular vaccines of the present invention contain one strain of A. salmonicida (MT004) grown under conditions of iron-limitation and one strain of A. salmonicida (MT423) grown under condition of iron-supplementation.

The Vibrio anguillarum (serotype 01) and V. anguillarum (serotype O₂) are different serotypes that are not cross-protective and therefore, for broad spectrum protection both antigens are can be included in the vaccine. Alternatively, or in combination, Vibrio ordalii can be employed.

### Administration

The vaccines of the present invention may be administered to fish by any of a number of means including by injection (e.g., intramucuscularly, or intraperitoneally), immersion, and/or through a delivery system for oral vaccination. Vaccinating fish by injection can be performed either with an adjuvant to increase the activity of the antigens, or without an adjuvant. Adjuvants include aqueous adjuvants, such as Alhydrogel or aluminum hydroxide, and oil adjuvants.

Mineral oil adjuvants are commonly employed in fish vaccines and are is included in the present invention. One such adjuvant is mannide oleate in a mineral oil solution. In a particular embodiment of this type, the vaccine comprises 70% mannide oleate in a mineral oil solution. Another mineral oil adjuvant of the present invention consists of white mineral oil, Span 80 [sorbitan monooleate], and Tween 80 [polyoxyethylene sorbitan monooleate]. In a particular embodiment, a vaccine comprises 80% of an adjuvant having the following formulation: 944 ml white mineral oil: 50.3 ml Span 80: 5.7 ml Tween 80.

Since mineral oil adjuvants generally cause damage to the fish at the site of injection (lesions, which have to be removed before sale) and they depress growth rates for a period of time, the present invention also provides non-mineral oil adjuvants. Synthetic non-mineral oil adjuvants include those commercially available from Seppic SA. Montanide, e.g., Montanide ISA563, Montanide ISA 575, Montanide ISA 711, and Montanide ISA 760. Montanide ISA 711 is essentially mannide oleate in an oil solution. Particular embodiments of a vaccine of the present invention comprise 50% of either Montanide ISA563, Montanide ISA 575, Montanide ISA 760 or 70% Montanide ISA 711.

Alternatively, vaccines can be applied by a long-term immersion bath. In one such embodiment, vaccination via an immersion bath is preceded by hyperosmotic treatment [see Huising et al., Vaccine 21:4178-4193 (2003)]. In another embodiment, a vaccine is administered by spraying the fish.

The present invention also includes orally-delivered vaccines. Generally, oral vaccines are prepared by either top-dressing the food with an antigen (e.g., by spray drying) or by incorporating the antigen in the food [see, e.g., Vinitnantharat et al., Adv. Vet. Med. 41:539-550 (1999)]. Other techniques include water-in-oil methods, bioencapsulation, microencapsulation incorporation into liposomes, incorporation in hollow feed prills, and incorporation into microparticle carriers, e.g., poly-lactide co-glycolide carrier particles [see, e.g., Singh et al., Expert Opin. Biol. Ther. 4(4):483-491 (2004)]. Yet another method entails expressing the antigen in algae.

Booster vaccines are also part of the present invention. In a particular embodiment, an oily emulsion oral booster vaccine comprising one or more antigens from the present invention is used after the primary vaccination. Preferably the oily emulsion is made up of water:oil in the range of 6:4 to 4:6. The level of free fatty acids should not be greater than 5% by weight of the oil and preferably no greater than 3%. Particular oils include whole fish body oil and neutral marine oil. The emulsifier is preferably food grade. Lecithin can be used as such an emulsifier, e.g., soya lecithin.

The emulsifier generally comprises from approximately 0.1% to approximately 5% by weight of the total emulsion. In a particular embodiment of this type, the oily phase of the emulsion is 47% v/v refined fish body oil plus 3% v/v lecithin (Bolec MT) which are mixed, sterilized with gamma irradiation and then blended, using an homogenizer. The aqueous antigen phase can be diluted with phosphate buffered saline [see, GB 2 255 909, PCT/GB9101828, WO/92/06599, the contents of which are hereby incorporated by reference in their entireties].

Injection vaccination is usually conducted on a commercial scale using a fixed dose automatic repeating syringe or an automatic injection vaccination machine. These methods are designed to deliver a fixed dose of usually 0.1 or 0.2 ml per fish. The vaccine is injected through the body wall into the intra-peritoneal cavity. It is also possible to immunize fish by injecting the vaccine into the dorsal sinus. Generally, fish are vaccinated by injection following anesthetization.

Immersion vaccination can be performed as follows: Dilute 1 liter of vaccine with 9 liters of clean hatchery water. Then Drain and weigh a netful of fish and dip fish in the diluted vaccine for 30 to 60 seconds ensuring that fish are totally immersed in the vaccine. After 30 to 60 seconds lift net, drain and return fish to holding tank. Repeat until 100 kg of fish have been dipped into 10 liters of diluted vaccine.

Oral vaccination can be performed as follows: A container of vaccine is brought to room temperature (20° C.) and then shaken prior to use. The vaccine is mixed with the fish feed so that the vaccine is coated onto the surface of the fish feed and adsorbed. The total vaccine dose should be fed over a 10 day period at 1/10 dose per fish per day.

### Vaccination Recipients

Salmonid rickettsial septicemia (SRS) was first observed in salmonids, which are the fish in the Salmonidae family, of the order Salmoniformes and of the class Osteichthyes. Salmonids are elongate bony fish with the last three vertebrae upturned, having a small adipose fin without fin rays between the dorsal fin and the tail. Many species of salmonids live in the sea, but enter fresh water to spawn. The Salmonidae family includes salmon, trout, char, and whitefish (see Table 1, below, which provides a non-exhaustive list of fish in the Salmonidae family).

TABLE 1
Salmonidae Family

| | |
|---|---|
| Coregonus clupeaformis | Lake whitefish |
| Coregonus hoyi | Bloater |
| Oncorhynchus keta | Chum salmon |
| Oncorhynchus gorbuscha | Pink salmon |
| Oncorhynchus kisutch | Coho salmon (silver salmon) |
| Oncorhynchus masou | cherry salmon (masou salmon) |
| Oncorhynchus nerka | Sockeye salmon |
| Oncorhynchus tshawytscha | King salmon (chinook salmon) |
| Prosopium cylindraceum | Round whitefish |
| Oncorhynchus clarki | Cutthroat trout |
| Oncorhynchus mykiss | Rainbow trout |
| Salmo salar | Atlantic salmon |
| Salmo trutta | Brown trout |
| Salmo trutta X S. fontinalis | Tiger hybrid-trout |
| Salvelinus alpinus | Arctic charr |
| Salvelinus confluentus | Bull trout |
| Salvelinus fontinalis | Brook trout |
| Salvelinus leucomaenis | Japanese charr (white spotted charr) |
| Salvelinus malma | Dolly varden (Miyabe charr) |
| Salvelinus namaycush | Lake trout |
| Thymallus thymallus | Grayling |

Reports of (SRS) and closely related Rickettsial syndrome afflicting fish as disparate as tilapia, white sea bass, rainbow trout, steelhead trout, grouper, Chilean sea bass, tiger puffers, red sea bream, blue-eyed plecostomus, striped bass, fluke, Atlantic cod, butter fish, ocean pout, spotted hake, summer and winter flounder, weakfish, yellowtail flounder, Windowpane flounder (Scophthalmus aquosus) cultured amberjack, three lined grunt, and blue eyed plecostomus indicates that the vaccines of the present invention may be used to vaccinate essentially any fish. Preferably the fish are in the Teleosti grouping of fish, i.e., teleosts. Both the Salmoniformes order (which includes the Salmonidae family) and the Perciformes order (which includes the Centrarchidae family) are contained within the Teleosti grouping.

Aside from the Salmonidae family and those included above, examples of potential vaccination recipients include the Serranidae family, the Sparidae family, the Chichlidae family, the Centrarchidae family, the three-Line Grunt (Parapristipoma trilineatum), and the Blue-Eyed Plecostomus (Plecostomus spp) of Tables 2 and 3, below.

TABLE 2

| | |
|---|---|
| TAXON NAME | COMMON NAME |

Some Members of the Serranidae Family

| | |
|---|---|
| Centropristis ocyurus | Bank sea bass |
| Centropristis philadelphicus | Rock sea bass |
| Centropristis striata | Black sea bass |
| Diplectrum bivittatum | Dwarf sandperch |
| Diplectrum formosum | Sand perch |
| Epinephelus flavolimbatus | Yellowedge grouper |
| Epinephelus morio | Red grouper |
| Serranus phoebe | Tattler |
| Serranus tortugarum | Chalk bass |

Some Members of the Sparidae family

| | |
|---|---|
| Archosargus | Sheepshead |
| probatocephalus | |
| Archosargus rhomboidalis | Sea bream |
| Calamus penna | Sheepshead porgy |
| Lagodon rhomboides | Pinfish |
| Pagrus Major | Red Sea bream |
| Sparus aurata | Gilthead Sea bream |
| Stenotomus chrysops | Scup |

Some Members of the Cichlidae family

| | |
|---|---|
| Aequidens latifrons | Blue acara |
| Cichlisoma nigrofasciatum | Congo cichlid |
| Crenichichla sp. | Pike cichlid |
| Pterophyllum scalare | Angel fish |
| Tilapia mossambica | Mozambique mouth breeder |
| Oreochromis spp | Tilapia |
| Sarotherodon aurea | Golden Tilapia |

TABLE 3
Some Members of the Centrarchidae family

| TAXON NAME | COMMON NAME |
|---|---|
| Ambloplites rupestris | Rock bass |
| Centrarchus macropterus | Flier |
| Elassoma evergladei | Everglades pigmy sunfish |
| Elassoma okefenokee | Okefenokee pigmy sunfish |
| Elassoma zonatum | Banded pigmy sunfish |
| Enneacanthus gloriosus | Bluespotted sunfish |
| Enneacanthus obesus | Banded sunfish |
| Lepomis auritus | Redbreast sunfish |
| Lepomis cyanellus | Green sunfish |
| Lepomis cyanellus X L. gibbosus | Green X pumpkinseed |
| Lepomis gibbosus | Pumpkinseed |
| Lepomis gulosus | Warmouth |
| Lepomis humilis | Orange-spotted sunfish |
| Lepomis macrochirus | Bluegill |
| Lepomis megalotis | Longear sunfish |
| Micropterus coosae | Shoal bass |
| Micropterus dolomieui | Smallmouth bass |
| Micropterus punctulatus | Spotted bass |
| Micropterus salmoides | Largemouth bass |
| Pomoxis annularis | White crappie |
| Pomoxis nigromaculatus | Black crappie |

The present invention may be better understood by reference to the following non-limiting Examples, which are provided as exemplary of the invention. The following examples are presented in order to more fully illustrate the preferred embodiments of the invention. They should in no way be construed, however, as limiting the broad scope of the invention.

### EXAMPLES

### Example 1

### Identification of Two 90 kDa Antigens

Potential antigens were identified by screening what was believed to be a P. salmonis DNA expression library with polyclonal antibodies raised against P. salmonis as follows:

DNA was isolated from P. salmonis and partially digested with the restriction enzyme, Sau3AI. The isolated DNA was then cloned into the vector λGEM-12 (Promega) at a BamHI site. λ phage structural proteins were then added and the phages were assembled. A phage library was produced containing 13,750 different phages. The library then was amplified in an E. coli host strain.

The library was next transferred to the pGEM-5zf (+) vector (Promega) via the NotI site. An E. coli strain was transformed with the library, grown in the presence of ampicillin, and selected for ampicillin resistance. Clones were screened by replica plating using nitrocellulose membranes. Following the lysis of the bacteria on the nitrocellulose membranes, the membranes were blocked with milk and then incubated with anti-P. salmonis polyclonal antibodies produced by immunizing rabbits with formaldehyde killed P. salmonis bacteria. Next, the membranes were washed and then developed with goat anti-rabbit-HRP conjugate. To detect recircularised plasmids that did not contain the inserts, the membranes were also exposed to X-gal. One isolated clone, designated 1057, appeared to express a 70-90 kDa protein. However, this putative 70-90 kDa protein was not purified, nor was it further characterized.

Initially, a Chilean strain of Yersinia ruckeri, a non-human enteric bacterium, was selected as the recombinant host cell to express the 70-90 kDa protein for possible use in a vaccine against SRS. This selection was based on the conventional wisdom that bacterial surface antigens are difficult to express in traditional bacterial vectors, particularly when the desired use for the recombinant vector would be as a vaccine antigen. Thus, it was believed the method of choice for expressing a P. salmonis surface antigen, as the 70-90 kDa protein was suspected of being, would be such a non-human enteric bacterium. Indeed, US Published Patent Application No. US20070207165 (A1) and WO2006037383(A1) specifically teach the use of such a recombinant Yersinia ruckeri vector to express P. salmonis proteins, and specifically exemplify the successful use of this vector to express the ^{Ps}p45 protein. In fact, this project was deemed completed when a recombinant Yersinia vector was isolated that was believed to encode the 70-90 kDa protein.

However, the isolated recombinant Yersinia vector was fraught with stability and expression problems. Indeed, it was never clear what this recombinant Yersinia vector expressed, and more importantly, never clear whether this recombinant Yersinia vector ever actually encoded a 70-90 kDa protein.

After considerable time and effort, this recombinant Yersinia vector was abandoned. The project was picked up again by sequencing the DNA of the parent clone 1057 clone. Surprisingly, rather than encoding one 70-90 kDa protein, it was found to contain 11 foreign open reading frames (ORFS). Furthermore, two of these open reading frames, ORF1 and ORF2, unexpectedly were found to encode approximately 90 kDa proteins. The sequences encoding these seemingly unrelated proteins were designated ORF1 (the p₁ 90 protein) and ORF2 (the p₂ 90 protein). The finding of 11 open reading frames, two of which encoded 90 kDa proteins, proves that the original assumption that the 1057 clone encoded a single P. salmonis antigen of about 90 kDa was incorrect.

The proteins encoded by ORF1 and ORF2 were further characterized. The protein encoded by ORF 1 (p₁ 90) was found to have six predictive hydrophobic regions, whereas the protein encoded by ORF 2 (p₂ 90) was found to have only a single hydrophobic region.

The DNA sequence encoding p₁ 90 (ORF1).

SEQ ID NO: 1:
ATGAAAAAGATAATTACAATGATGTTATTGGTGTTATCACTTGTGTTGGT
CGCTTGTACCCCAAGTGAAGAACCACCAACTACAGTGCCAGATGTTGAAT
CCATCGAATTTAATATGACTTCAACGACTGTAGCACCAGGTGAACATACA
CTAGTTGCAAAAGCATTACCTGAAGGATCTAATCAACAAATTAGATTTAG
TATTCAAGGTATTGTATCTGGTGTATCCATTACGGGTGATAAGTTAAATG
TTGGTAATGCTGTTGAAGATGGTATGAAATTTACAGTCGTAGCAACATCT
GTATATGATCCAACAATTCGTGCAACACTAGAGTTTACAGTTGTAAATGT
TGGTGTTGAAGTTGTTGAAATTAGAACAGAAGAAGAACTACGTGCAATTC
ATACAAATGAAGGTGGTTTATCATTATCTTATGTATTAATGAATGATATT
GAACTAACAGCTCCATGGACACCAATTGGTATTGCTGAAGTTGAAACTGA
TTCTGGGCAAATCATTCCAGGTACGCCATTTAATGGTATCTTTAATGGAA
ATGGTTTTACAATTAGTGGCATATTAGTTGAAAGTGAAGAACCATTATTT
AATGCAGGATTCTTTGCTCAAATTGGTGCAACTGCAATTGTTAAGAATAC
AACATTTGAAGGTATAGTAAATGCAACCGGATGGTCCGGTGGTATCGCAG
GTATTAACGAAGGTTTAATAGAAAATGTTGTATCTAATGTAAGAGTTACT
GTAACAGGTACATCCGCAGGTTCGCTAGTATCTGTTAATAGAGGTTTAAT
CCAATATGCTTACGGTATTGGTAAAGTTGTTAGTGAAACAAACCCTAATA
CATCAGGTCGTTCTGCTGGTTTAGTGGTTGCTAATGATGGATCAATGATT
GAAGTGTATGGTGACTATCAAGCACTTGGAACACCTAACTATACAGCATT
TAGTCCATCAACAAACCCAATGTATATGTTACCTACAGTAGATATGAAAA
CATCTTCAACTTGGGCTTCATTTGATGCAGATGTTTGGTATATTGAAAAT
GGTACATATCCATTATTAAAACATGAAGGATTCGTTCCACCAGTGATCGT
TCCTGAATTAGGTATTACAATTAAAAATACTGAGTTAAATCATGATGTTG
AAGTATCAAGTGAACTACAAATAAATGCAGAAGTCATTAACCCAGAAGGT
AGTGAAGTTATTGTTTATGCACTTAAAGAAGCAGTAGCAGGTGTAGCAAT
TAGTGAAACAGGTTTAGTTACATTTGATATCACTACAATTGCTGCTAACT
TCTCATTTACAGTAGTAGTGACAATTGATGGTACTGAAGTTAGTGCTGAA
AAAACATTTACAGGCGTATATAACCCTGAAATTGTAGATGATACAGTGTA
TATTGAAACAGAAACACAATTATTAAACTTACTTGCTGGACAAACAAACC
CAGACAATTTAAGTAAAACATTTGTATTATTAAATGATATTGTCTTAACT
TCTAATTGGACAGCAATTGGTATTGCACCAAATGAAGACGAAGGTATTGT
AGGTGTTCCATTTACAGGTGTATTTGATGGTCAAGGCTATAAGATCTCAG
GTATTAGTATGCCAGGTGGTGGATGGAATAAAGGTTTCTTTGGATATATT
GGAACAACTGGTGTTGTTAAAAACACACACTTTGAAGGTAATCTAGAAGC
AAACGCATGGTCAGGTGCACTTGCAGCAAATAACTCAGGTACTATTCAAG
ATGTAGTTGTTGATATTGAAGTATATGTCTGGGGTAATAATGGTGGCGCA
ATCGTTGAACATAACCATGGTCTACTTAAAAATATTGTCGTATTAGGTAA
AGCTGTATCAGATAGTGGTCCTACAGCAGTTGGACTGGTTGTTACTAACT
TTGGTACTTTAGAAAATGTATTTGCTAACGCAGATACAGTAGGTACAGCA
AACTTAGTATCTAATGGTGCTCTCGCTGATGATGGTAAACACATTATTAG
TGCCCAAGACTTTGTTAAAGCTACAACTTATGCAAACTTTGATAGCGCAA
TCTGGTTAATCGTAGATGGCCAAGTACCTGTATTAATTAATGAAGATACA
GTATTACCTGAAACAGTAGTTTATATTGAAACAGAAGCAGAATTATTAAG
CCTACTTGCTGGTCAAGTAGATCCAGAAGCATTATCAAAAACATACAAAC
TTAAAAATGATATCGTTCTAACTTCTAATTGGACAGCAATTGGTATTGCA
CCAAATGAAGACGAAGGTATTGTAGGTGTTCCATTTACAGGTGTATTTGA
TGGTCAAGGCTATAAGATCTCAGGTATTAGTATGCCAGGTGGCGGATGGA
ATAAAGGTTTCTTTGGTTACATTGGAACAACGGGTGTTGTTAAAAATACA
CACTTTGAAGGTAATATTGAAGCAAACGCATGGTCAGGTGCACTTGCAGC
AAATAACTCGGGTACTATTATGGATGTTGTAGTAGACATTGAAGTATATG
TCTGGGGTAATAATGGTGGTGCAATTGTTGAACATAACCACGGTTTACTT
AAAAATATTATCGTCTTAGGTAAAGCTGTATCAGATGGTGGTCCTACAGT
AGTTGGACTAGTTGTTACTAACTTTGGTACACTAGAAGATGTATATGCAA
ATGTTGACACAGTAGGTACTTTAAACTTAGTATCATTTGGTAGCGTAGCA
GATGATGGTACACACATTATTAGTGCTTCAAACTTTGTTAAAGCAGAAAC
TTATGCAAACTTCTCAAGTGATGTTTGGACAATTATTGATGGTAGCACCC
CTGTATTAAAACAAGCATAA

The amino acid sequence of p₁ 90 (ORF1):

SEQ ID NO: 2
MKKIITMMLLVLSLVLVACTPSEEPPTTVPDVESIEFNMTSTTVAPGEHT
LVAKALPEGSNQQIRFSIQGIVSGVSITGDKLNVGNAVEDGMKFTVVATS
VYDPTIRATLEFTVVNVGVEVVEIRTEEELRAIHTNEGGLSLSYVLMNDI
ELTAPWTPIGIAEVETDSGQIIPGTPFNGIFNGNGFTISGILVESEEPLF
NAGFFAQIGATAIVKNTTFEGIVNATGWSGGIAGINEGLIENVVSNVRVT
VTGTSAGSLVSVNRGLIQYAYGIGKVVSETNPNTSGRSAGLVVANDGSMI
EVYGDYQALGTPNYTAFSPSTNPMYMLPTVDMKTSSTWASFDADVWYIEN
GTYPLLKHEGFVPPVIVPELGITIKNTELNHDVEVSSELQINAEVINPEG
SEVIVYALKEAVAGVAISETGLVTFDITTIAANFSFTVVVTIDGTEVSAE
KTFTGVYNPEIVDDTVYIETETQLLNLLAGQTNPDNLSKTFVLLNDIVLT
SNWTAIGIAPNEDEGIVGVPFTGVFDGQGYKISGISMPGGGWNKGFFGYI
GTTGVVKNTHFEGNLEANAWSGALAANNSGTIQDVVVDIEVYVWGNNGGA
IVEHNHGLLKNIVVLGKAVSDSGPTAVGLVVTNFGTLENVFANADTVGTA
NLVSNGALADDGKHIISAQDFVKATTYANFDSAIWLIVDGQVPVLINEDT
VLPETVVYIETEAELLSLLAGQVDPEALSKTYKLKNDIVLTSNWTAIGIA
PNEDEGIVGVPFTGVFDGQGYKISGISMPGGGWNKGFFGYIGTTGVVKNT
HFEGNIEANAWSGALAANNSGTIMDVVVDIEVYVWGNNGGAIVEHNHGLL
KNIIVLGKAVSDGGPTVVGLVVTNFGTLEDVYANVDTVGTLNLVSFGSVA
DDGTHIISASNFVKAETYANFSSDVWTIIDGSTPVLKQA

The DNA sequence encoding p₂ 90 (ORF2);

SEQ ID NO: 3:
ATGCATTTGGTGTTGTTGCTAAAAAGTAAAAAGGACAAATATATGAAAAA
AATAAGCTTAATAATGATTTTTCTGCTTTCTATCCTATTGGTAAGTTGTG
TAGAAAAAGAAGAACCAAAATTTGATCCAGATAAATATCTAGATTTAGAG
AATATTGTATTTGATGATTTTGATAACGGAATTGACCCGAATATGTGGGT
TATTGGTAATAGTAAGTGGGGTGTAGGTAATGGTGGTGTCATCTATGAAA
ATGTCCATTACACAAATGACGGTATTGTAGTTCTTCAAACCAATGGTGAC
TTGTATGATGGTCCACTTCGCGGTATTGGTAATACCCATGGCAGACGTAC
AGGTGCAATGATTACAACAAGAGAAGCACTAGGTCCTGGTAGATTTGAAG
TACGTATGCGTATTATGCCACGTTTTGGTTCAACTACTGCTATGTGGACT
TACTATTATGATAATGGTATGAACCATGAAATAGATATCGAAAGTAACGT
TGAAAATGACTTTAGAAAAGTATGGACTACAAACTGGATTAGTTTAACAG
AATATAGTACTGTGTCTAATACCTTAGATTTTGCACAAAATGATTTTGAA
TGGCGTACATACCGTTTTGACTGGTTTACAGATCCAAAACGCATTGATTA
TTATATTGATGAAGTATTAGTTTCATCACAATCTTCTTATGTACCAGATC
ATGCAGGAGAATTTAATATTGGTAATTGGTTTCCAGATGCTTGGGCAGGT
GTACCTGATTTTGAAACAGACTATACCTATGTAGACTGGTTCAAATATAC
ACCATTTAAAGAACAACCATATACACCAACACCGGCAAATAATCAAAGTC
CTGCAAACTTCTATCCATCAGAACCAATTGAACATCCAATAGCAAACCTC
ATTTCAAATGCAGGTTTTGAAACAGATGCTCCAGCTTGGCGTTATCCTGT
AACTAGTGGTGTGGAACTACTAGAAGGTGAAGGTTTAAACGGATCAAGAG
GAATCTTTGTTCCACAAAATGATATTGCATATCAATTTGTCACAGGATTA
GATGAAACCTTTGAAATGACATTTAGTGCACATGCAAAACTACCTTTAAA
TGGTAGTGGATATGTTTTATTAGAGTTCTACCCAGCAGAGACACAAAAAA
TTGATCAGTATATGATTGAGTTTAACTCAAGCGATGAAGATTTTATAGCA
GATACATTCTATGGTAAAGAATTTACCTTTAATGTACCTCTAGGAACTAA
ACGTGTTGAAGTGTCTTTAATTGGAGGAGATTCTGGTATATACTTCGATG
ATTTATTCTTTAACCTAACTAAAAAACCCAGGCCCGAAATTGTAGAAGAA
GGTGATGATGTGCAACGTTTAAACATAGATTTTAAAAATGGTATTGACTC
CAATGTTTGGGCAGTTGCAAATCAACGTTGGGGAGGTACACATCATGGTG
GTGTAATCTTCCAAAACGTACACTACACAGAAGAAGGTAATTTACTCATT
CAAGCCAATGGTGATTACTATGAAGGTCCATTAAAAGGTGTTGAACAAAA
TAATGGAAAACGCACTGGGGGAGCTATCTATACTAAAGAAGCATTTGGCC
CAGGATCTTTTGAAGTAAAAGCTAAAATCATGCCACGTTTTGGGGCAACA
ACAGCATTTTGGACATTTAACTACTTAGATGGTATTAATAGTGAAATTGA
TTTTGAGTTTAACGTAGGCAATGATTTTAGTACAGTTTGGTTAACCAACT
GGTTAACCGAAACAAACTATAACAACTACACCCATCAAATGGATAGTTTC
CATAATGATGGAAACTGGCATATATACCGTTTTGAATGGCATACACTACC
GACGCCTCATATTAAATACTTTATCGATGGCAAACTTGCATATACAGAAC
ATACTAAAGTTCCAACGATGTCTGCAAGATACTGGATTGGTGTATGGTTT
CCAAATAACTGGGCAGGAGATCCAAACTTTGAAACAGATTATTTAGAAGT
TGAATATTTCAAATATGAGTCATTCCCGGATCATCCGTATGTTGTTGGTC
CAACTGGGGCATCCTCTCCAACAGCATTTTACCCAACAGCGCCAATAAAA
AAACCAGTTTCTAACCTTTTACCACACGGTAATCTAGATTATGAAACAGG
TTATATGTTAACAGGGGATGCAGTGATTTCAAATGGTGAATTGAAAACTG
GTTTACTAGGCAGTGCTGAGTCTCTTATTACAGGGTTAAATGATGCCTTT
GAACTTACATTAAAGCTTAAAGCAAAAGCCTCAAATAACGCAACCGTGCG
CATTGAGTATTTAGATAAGGATTTAAATGTGATAAGTGGTGAAGATATTA
TTGTATCAAACTTAAACGCGAATACATTTACAAACTTTACATCCGTAATT
AATCTAGTGGAAGGCACTAGAGCCATCAATGTGATTTTTGAGGGAACAAA
TATCACATATGATGACTTATTTATAAATTTAACACACAAGGTGAATTGA

The amino acid sequence of p₂90 (ORF2):

SEQ ID NO: 4
MHLVLLLKSKKDKYMKKISLIMIFLLSILLVSCVEKEEPKFDPDKYLDLE
NIVFDDFDNGIDPNMWVIGNSKWGVGNGGVIYENVHYTNDGIVVLQTNGD
LYDGPLRGIGNTHGRRTGAMITTREALGPGRFEVRMRIMPRFGSTTAMWT
YYYDNGMNHEIDIESNVENDFRKVWTTNWISLTEYSTVSNTLDFAQNDFE
WRTYRFDWFTDPKRIDYYIDEVLVSSQSSYVPDHAGEFNIGNWFPDAWAG
VPDFETDYTYVDWFKYTPFKEQPYTPTPANNQSPANFYPSEPIEHPIANL
ISNAGFETDAPAWRYPVTSGVELLEGEGLNGSRGIFVPQNDIAYQFVTGL
DETFEMTFSAHAKLPLNGSGYVLLEFYPAETQKIDQYMIEFNSSDEDFIA
DTFYGKEFTFNVPLGTKRVEVSLIGGDSGIYFDDLFFNLTKKPRPEIVEE
GDDVQRLNIDFKNGIDSNVWAVANQRWGGTHHGGVIFQNVHYTEEGNLLI
QANGDYYEGPLKGVEQNNGKRTGGAIYTKEAFGPGSFEVKAKIMPRFGAT
TAFWTFNYLDGINSEIDFEFNVGNDFSTVWLTNWLTETNYNNYTHQMDSF
HNDGNWHIYRFEWHTLPTPHIKYFIDGKLAYTEHTKVPTMSARYWIGVWF
PNNWAGDPNFETDYLEVEYFKYESFPDHPYVVGPTGASSPTAFYPTAPIK
KPVSNLLPHGNLDYETGYMLTGDAVISNGELKTGLLGSAESLITGLNDAF
ELTLKLKAKASNNATVRIEYLDKDLNVISGEDIIVSNLNANTFTNFTSVI
NLVEGTRAINVIFEGTNITYDDLFINLTHKVN

The DNA sequences encoding ORF 1 and ORF 2 were then optimized for E. coli codon usage, avoiding internal TATA boxes, chi sites and ribosomal entry sites, and AT or GC rich sequences. Synthetic genes were made and cloned via pPCR-Script into pEGT1 (the vector was constructed by EGT, and contains a T7/lac promoter, f1 origin, is Kanamycin resistant, (KanR), and Hok-Sok). Western blot analyses with anti-peptide antibodies to ORF 1 and ORF 2 confirmed expression of approximately 90 kDa proteins from both ORF 1 and ORF 2.

The E. coli optimized DNA sequence encoding p₁ 90 (ORF1); ORF1 sequences in pEGT1 (EGT optimised sequences):

SEQ ID NO: 5
ATGAAAAAAATCATCACCATGATGCTGCTGGTTCTGAGCCTGGTTCTGGT
GGCGTGTACCCCGTCTGAAGAACCGCCGACCACCGTTCCGGATGTGGAAA
GCATTGAATTTAACATGACCAGCACCACCGTGGCACCGGGCGAACATACC
CTGGTGGCGAAAGCGCTGCCGGAAGGCAGCAACCAGCAGATTCGTTTTAG
CATTCAGGGCATTGTGAGCGGCGTGAGCATTACCGGCGATAAACTGAACG
TGGGCAACGCCGTGGAAGATGGCATGAAATTTACCGTTGTGGCGACCAGC
GTGTATGACCCGACCATTCGTGCCACCCTGGAATTTACCGTGGTTAACGT
TGGCGTGGAAGTGGTGGAAATTCGTACCGAAGAAGAACTGCGCGCGATTC
ATACCAACGAAGGCGGCCTGAGCCTGAGCTATGTGCTGATGAACGATATT
GAACTGACCGCCCCGTGGACCCCGATTGGCATTGCCGAAGTGGAAACCGA
TAGCGGCCAGATTATTCCGGGCACCCCGTTTAACGGCATTTTTAACGGCA
ACGGCTTTACCATTAGCGGCATTCTGGTGGAAAGCGAAGAACCGCTGTTT
AACGCCGGCTTTTTTGCCCAGATTGGCGCCACCGCCATTGTGAAAAACAC
CACCTTTGAAGGCATTGTGAACGCCACCGGCTGGAGCGGCGGCATTGCCG
GCATTAACGAAGGCCTGATTGAAAACGTTGTTAGCAACGTTCGTGTGACC
GTGACCGGCACCAGCGCCGGTAGCCTGGTGAGCGTGAACCGTGGCCTGAT
TCAGTATGCCTATGGCATTGGCAAAGTGGTGAGCGAAACCAACCCGAACA
CCAGCGGTCGTAGCGCCGGTCTGGTGGTGGCGAACGATGGCAGCATGATT
GAAGTGTATGGCGATTATCAGGCGCTGGGCACCCCGAACTATACCGCCTT
TAGCCCGAGCACCAACCCGATGTATATGCTGCCGACCGTGGATATGAAAA
CCAGCAGCACCTGGGCGAGCTTTGATGCCGATGTGTGGTATATCGAAAAC
GGCACCTATCCGCTGCTGAAACATGAAGGCTTTGTGCCGCCGGTTATTGT
GCCGGAACTGGGCATTACCATTAAAAACACCGAACTGAACCATGATGTGG
AAGTGAGCAGCGAACTGCAGATTAACGCCGAAGTGATTAACCCGGAAGGT
AGCGAAGTTATTGTTTATGCCCTGAAAGAAGCGGTGGCGGGCGTTGCCAT
TAGCGAAACCGGCCTGGTGACCTTTGATATTACCACCATTGCGGCGAACT
TTAGCTTTACCGTGGTGGTGACCATTGATGGCACCGAAGTGAGCGCCGAA
AAAACCTTTACCGGCGTGTATAACCCGGAAATTGTGGATGATACCGTCTA
TATCGAAACCGAAACCCAGCTGCTGAACCTGCTGGCGGGCCAGACCAACC
CGGATAACCTGAGCAAAACCTTTGTGCTGCTGAATGACATTGTGCTGACC
AGCAACTGGACCGCCATTGGTATTGCCCCGAACGAAGATGAAGGTATTGT
TGGCGTTCCGTTTACCGGTGTGTTTGATGGCCAGGGCTACAAAATTAGCG
GTATTAGCATGCCGGGTGGCGGCTGGAACAAAGGCTTTTTTGGCTATATC
GGCACCACCGGCGTGGTGAAAAATACCCATTTCGAAGGTAACCTGGAAGC
GAACGCCTGGTCTGGCGCCCTGGCGGCGAACAACAGCGGCACCATTCAGG
ATGTGGTGGTGGATATCGAAGTGTATGTTTGGGGCAACAACGGCGGTGCC
ATTGTGGAACATAACCATGGCCTGCTGAAAAACATTGTGGTGCTGGGTAA
AGCGGTGAGCGATAGCGGTCCGACCGCCGTGGGTCTGGTGGTTACCAACT
TTGGCACCCTGGAAAACGTGTTTGCCAACGCCGATACCGTGGGCACCGCC
AACCTGGTGAGCAACGGTGCCCTGGCGGATGATGGCAAACACATTATCAG
CGCCCAGGATTTTGTGAAAGCGACCACCTATGCCAACTTTGATAGCGCCA
TTTGGCTGATTGTGGATGGCCAGGTGCCGGTTCTGATTAACGAAGATACC
GTGCTGCCGGAAACCGTGGTGTATATTGAAACCGAAGCGGAACTGCTGTC
TCTGCTGGCGGGTCAGGTGGATCCGGAAGCGCTGTCTAAAACCTACAAAC
TGAAAAACGATATCGTGCTGACCTCTAACTGGACGGCGATCGGCATCGCT
CCGAATGAAGATGAGGGCATCGTCGGCGTCCCGTTCACCGGCGTGTTCGA
CGGTCAGGGTTATAAAATTTCTGGCATTTCTATGCCGGGTGGTGGTTGGA
ATAAAGGTTTCTTCGGTTACATTGGCACCACCGGTGTTGTTAAAAACACT
CACTTTGAGGGTAATATTGAAGCGAATGCCTGGAGCGGCGCTCTGGCCGC
CAACAACTCTGGCACCATTATGGATGTTGTTGTCGATATTGAAGTTTACG
TGTGGGGCAATAATGGTGGCGCCATCGTTGAACACAATCACGGTCTGCTG
AAAAATATCATTGTTCTGGGTAAAGCCGTTTCTGATGGCGGTCCGACGGT
GGTGGGCCTGGTTGTGACGAATTTCGGCACGCTGGAAGATGTGTATGCCA
ATGTTGATACCGTTGGCACCCTGAATCTGGTGAGCTTTGGCAGCGTGGCC
GATGATGGCACCCATATCATTAGCGCCAGCAACTTTGTTAAAGCGGAAAC
CTATGCCAATTTTAGCAGCGATGTGTGGACCATTATTGATGGCAGCACCC
CGGTGCTGAAACAGGCGTAA

The E. coli optimized DNA sequence encoding p₂ 90 (ORF2); ORF2 sequences in pEGT1 (EGT optimised sequences). Note the corresponding E. coli optimized p₂ 90 amino acid sequence begins with an N-terminal histidine instead of the N-terminal proline for the naturally occurring p₂ 90 protein.

SEQ ID NO: 6
ATGCATCTGGTGCTGCTGCTGAAAAGCAAAAAAGATAAATACATGAAAAA
AATCAGCCTGATCATGATTTTTCTGCTGTCTATTCTGCTGGTGAGCTGTG
TGGAAAAAGAAGAACCGAAATTCGATCCGGATAAATACCTGGATCTGGAA
AACATCGTTTTCGATGATTTCGATAACGGCATTGATCCGAACATGTGGGT
GATTGGCAACAGCAAATGGGGCGTGGGCAACGGCGGCGTGATTTATGAAA
ACGTCCATTACACCAACGATGGCATTGTGGTGCTGCAGACCAACGGCGAT
CTGTATGATGGCCCGCTGCGTGGCATTGGCAACACCCATGGCCGTCGTAC
CGGCGCCATGATTACCACCCGTGAAGCGCTGGGTCCGGGCCGTTTTGAAG
TTCGTATGCGCATTATGCCGCGTTTTGGCAGCACCACCGCCATGTGGACC
TATTATTATGATAACGGCATGAACCACGAAATTGATATCGAAAGCAACGT
GGAAAACGATTTTCGTAAAGTTTGGACCACCAACTGGATCAGCCTGACCG
AATATAGCACCGTGAGCAACACCCTGGATTTTGCCCAGAACGATTTTGAA
TGGCGTACCTATCGTTTTGATTGGTTTACCGATCCGAAACGTATCGATTA
CTACATTGATGAAGTGCTGGTGAGCAGCCAGAGCAGCTATGTGCCGGATC
ATGCCGGCGAATTTAACATTGGCAACTGGTTTCCGGATGCCTGGGCAGGC
GTTCCGGATTTTGAAACCGATTATACCTACGTGGATTGGTTTAAATACAC
CCCGTTTAAAGAACAGCCGTATACCCCGACCCCGGCGAATAACCAGAGCC
CGGCGAACTTTTATCCGAGCGAACCGATTGAACATCCGATTGCCAACCTG
ATTAGCAACGCCGGCTTCGAAACCGATGCCCCGGCATGGCGTTATCCGGT
GACCAGCGGCGTGGAACTGCTGGAAGGCGAAGGCCTGAACGGCAGCCGTG
GCATTTTTGTGCCGCAGAACGATATTGCCTATCAGTTTGTGACCGGCCTG
GATGAAACCTTTGAAATGACCTTTAGCGCCCATGCCAAACTGCCGCTGAA
CGGTAGCGGCTATGTGCTGCTGGAATTTTATCCGGCGGAAACCCAGAAAA
TTGACCAGTATATGATCGAATTCAACAGCAGCGATGAAGATTTTATCGCC
GATACCTTCTATGGCAAAGAATTTACCTTTAACGTTCCGCTGGGCACCAA
ACGTGTGGAAGTGAGCCTGATTGGCGGCGATAGCGGCATTTATTTTGACG
ACCTGTTCTTCAACCTGACCAAAAAACCGCGTCCGGAAATTGTGGAAGAA
GGCGACGACGTTCAGCGTCTGAACATTGATTTCAAAAACGGCATCGATAG
CAACGTGTGGGCGGTGGCGAATCAGCGTTGGGGCGGCACGCATCATGGCG
GTGTGATTTTTCAGAACGTTCACTATACCGAAGAAGGCAACCTGCTGATT
CAGGCGAACGGCGATTATTATGAAGGTCCGCTGAAAGGCGTTGAACAGAA
CAACGGCAAACGTACCGGCGGTGCCATTTATACCAAAGAAGCGTTTGGCC
CGGGTAGCTTTGAAGTGAAAGCGAAAATCATGCCGCGCTTTGGTGCCACC
ACGGCGTTTTGGACCTTTAACTATCTGGATGGCATCAACAGCGAAATCGA
TTTTGAATTCAACGTGGGCAACGATTTTAGCACCGTGTGGCTGACCAACT
GGCTGACCGAAACCAACTATAACAACTACACCCATCAGATGGATAGCTTT
CATAACGATGGCAACTGGCATATTTATCGCTTTGAATGGCATACCCTGCC
GACCCCGCATATTAAATACTTCATCGACGGCAAACTGGCGTATACCGAAC
ATACCAAAGTGCCGACCATGAGCGCCCGTTATTGGATTGGCGTGTGGTTT
CCGAACAACTGGGCGGGTGATCCGAACTTTGAAACCGACTATCTGGAAGT
GGAATACTTCAAATACGAAAGCTTTCCGGATCATCCGTATGTTGTTGGCC
CGACCGGTGCCTCTAGCCCGACCGCCTTTTATCCGACCGCCCCGATTAAA
AAACCGGTGAGCAACCTGCTGCCGCATGGCAACCTGGATTATGAAACCGG
CTATATGCTGACCGGCGATGCCGTGATTAGCAATGGCGAACTGAAAACCG
GCCTGCTGGGCAGCGCCGAAAGCCTGATTACCGGCCTGAACGATGCCTTT
GAACTGACCCTGAAACTGAAAGCGAAAGCGAGCAACAACGCCACCGTTCG
TATTGAATACCTGGATAAAGATCTGAACGTTATCAGCGGCGAAGATATTA
TTGTGAGCAATCTGAACGCCAACACCTTTACCAACTTTACCAGCGTGATT
AACCTGGTTGAAGGCACCCGTGCCATTAACGTTATTTTCGAAGGCACGAA
CATTACCTATGATGACCTGTTTATTAACCTGACCCACAAAGTGAACTAAT
AAGTCGACG

### Example 2

### Expression of the Two 90 kDa Antigens

### Upstream Process

Batches of the two 90 kDa antigens were produced,

The strains used were as follows:

E. coli HMS174(DE3)/pEGT1/AL-ORF1-90 kDa (p₁ 90)

E. coli HMS174(DE3)/pEGT1/AL-ORF2-90 kDa (p₂ 90)

The strains were grown in shake-flasks. Each batch was prepared as a pool of 5 shake flasks.

The protocol was as follows:
1. 50 μl of a glycerol stock was grown in 200 mL of YES medium [30 g/l yeast extract, 5 g/l NaCl], supplemented with kanamycin 100 mg/l [500 ml medium in a 2 L shake-flask].
2. The culture was incubated at 37° C., with an agitation of 270 rpm.
3. When the OD₆₀₀ reached 2.1, protein expression was induced by the addition of 1 mM isopropyl-β-D-thiogalactopyranoside (“IPTG”).
4. The final OD₆₀₀ was around 2.2 for the ORF1-strain, and 1.7 for the ORF2-strain.
5. At the end of the culture, the pellets were harvested by centrifugation (5000 g, 30 min., 4° C.) and the supernatant discarded.
6. Pellets were collected and stored at −20° C.
7. Cell breakage: Pellets were resuspended in 115 mL 20 mM Tris buffer pH 7 for the ORF1-antigen and in 85 ml 20 mM Tris buffer pH 7 for the ORF2-antigen. A French Press was used to break the cells and 2 cycles were performed (40K; 1000 PSI; room temperature, all samples are keep on ice). Volumes collected were 120 ml for the ORF1-antigen and 75 ml for the ORF2-antigen. The samples were stored at −20° C.

### Example 3

### Purfication of Two 90 kDa Antigens

### Downstream Process

120 ml of the ORF1 antigen (p₁ 90) and 75 ml of the ORF2 (p₂ 90) antigen samples prepared according to Example 2 were semi-purified using a 300 kDa ultrafiltration. The retentates were concentrated to 50 ml and then diafiltered against 5 volumes of 20 mM Tris, pH 7.5. Both antigens were found to be in the retentate fractions forming large aggregates. The two antigens were then formulated by diafiltration with PBS buffer (137 mM NaCl, 2.7 mM KCl, 8 mM Na₂HPO₄.12H₂O, 1.5 mM KH₂PO₄, pH=7.45) using a 300 kDa molecular weight cut-off ultrafiltration membrane.

### Example 4

### Formulation of Vaccines

125 ml of the 300-kDa retentates, as produced by Example 3, were then diafiltrated against 5 volumes of PBS buffer (137 mM NaCl, 2.7 mM KCl, 8 mM Na₂HPO₄.12H₂O, 1.5 mM KH₂PO₄, pH=7.45). The retentates containing the respective ORF-1 and ORF-2 90-kDa-antigens were collected and stored at −20° C. The antigens were then sterile filtered through 0.2 μm filters prior to vaccine blending.

The final protein concentration for the two antigens preparations were 0.089 mg/ml for ORF1 and 0.015 mg/ml of ORF2. SDS PAGE gels were run and scanned in order to determine the ratio of the 90 kDa antigens to total protein. The ORF1 antigen was found to be 25.7% of the total protein and the ORF 2 antigen was found to be 8.3%. Final concentrations of the specific antigens were therefore 0.023 mg/ml for ORF 1 (p₁ 90) and 0.0012 mg/ml for ORF 2 (p₂ 90).

The vaccines were blended as follows:

The oil based adjuvant Montanide ISA 711 (Seppic) was used in a 70:30 (adjuvant:antigen) ratio.

| | |
|---|---|
| Blend 1 (90 kDa ORF1) | 15 ml (0.345 mg) antigen, 35 ml adjuvant |
| Blend 2 (90 kDa ORF2) | 15 ml (0.018 mg) antigen, 35 ml adjuvant |
| Blend 3 (PBS control) | 15 ml PBS, 35 ml adjuvant |

Following mixing of the aqueous and oil phases, each of the respective vaccines was homogenised by serial passages through a syringe.

### Example 5

### Laboratory Efficacy Trial of Vaccination of Atlantic Salmon (Salmo Salar) Against SRS

The vaccines prepared according to Example 4 above, were tested for efficacy employing a challenge with P. salmonis at VESO Vikan, Norway.

Atlantic salmon (Salmo salar L., AquaGen Standard) were vaccinated at an average weight of 16.1 g in fresh water. Water temperature was 12° C. to 15° C.±1° C. during the immunisation period and 15° C.±1° C. during the P. salmonis challenges. The water flow rate was 0.8 l/kg fish per min. The fish density was a maximum of 40 kg/m³.

Fish were acclimatised for 18 days after arrival at VESO Vikan. 105 fish were anesthetized and injected with 0.1 ml of each vaccine per fish, and a control group of 105 fish was anesthetized and injected with 0.1 ml of physiological saline with 0.9% NaCl per fish. The fish were tagged with ink and by fin cutting. The test groups and the control group were pooled after vaccination. 12 days before challenge the fish were sorted into two parallel tanks, with 50 fish per group per tank. 10 days before challenge the water temperature was raised to 15° C.

Laboratory efficacy trial—challenge 48-52 fish from each group were challenged with P. salmonis by intraperitoneal injection in two duplicate tanks 8 weeks after vaccination. The same challenge dose was used in both tanks (0.1 ml 1:1000 dilution per fish, and the dose was determined following prechallenge experiments). The temperature in the rearing water was 15° C. during the challenge. The challenge material was grown at The National Veterinary Institute, Oslo and was kept at −75° C. until challenge. On the day of challenge, the challenge material was dose adjusted according to the results obtained in the pre-challenge. The challenge material was diluted in PBS with 1.3% NaCl.

Relative percentage survival (RPS₆₀) was calculated on the day that control group mortality equalled exactly 60% or, if inappropriate, at the time corresponding to 60% control group mortality (t60) according to the expression:

$${RPS}_{60} = ( 1 - ( \frac{{MV}_{60} \times 100}{{n}_{v} \times 60} ) ) \times 100$$

In cases where control-group mortality did not equal exactly 60% on any given day of the trial, cumulative mortality of vaccinates at the time corresponding to 60% control group mortality was estimated from the expression.

$${MV}_{60} = {MV}_{60 - 1} + ( \frac{( ( 0.6 \times {n}_{c} ) - {MC}_{60 - 1} ) \times ( {MV}_{60 + 1} - {MV}_{60 - 1} )}{( {MC}_{60 + 1} - {MC}_{60 - 1} )} )$$

The following definitions apply to the parameters of the first and second expressions, above.
MV₆₀=cumulative mortality of vaccinates at 60% cumulative control group mortality.
MC₆₀₋₁=number of mortalities in control group on last day before cumulative mortality rate in control group exceeds 60%.
MC₆₀₊₁=number of mortalities in control group on first day after cumulative mortality rate in control group has exceeded 60%.
MV₆₀₋₁=number of mortalities in vaccinated group on last day before cumulative mortality rate in control group exceeds 60%.
MV₆₀₋₁=number of mortalities in vaccinated group on first day after cumulative mortality rate in control group has exceeded 60%.
n_{c}=total number of fish in control group.
nᵥ=total number of fish in vaccinated group
RPS₆₀=Relative percentage survival at 60% control group mortality.
   RPS₆₀ was subsequently calculated from the first expression, with the following results.

### Results

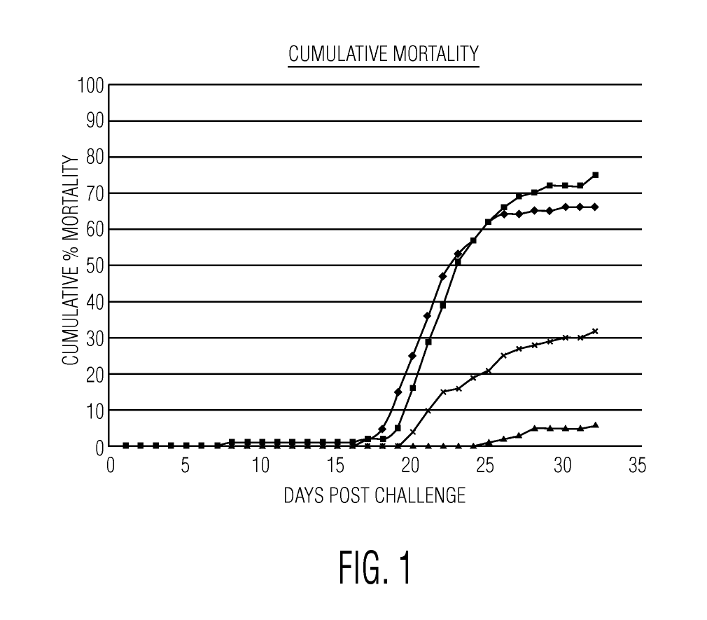

Results for the challenge are summarized by FIG. 1, and by the tables below. As can be seen in FIG. 1, the challenged animals vaccinated by saline control (diamonds) or adjuvant control (squares) exhibited up to about 75% mortality by days 25-30 post challenge. In contrast, animals vaccinated with the ORF-1 antigen (p₁ 90), denoted by “X” and the ORF-2 antigen (p₂ 90) denoted by triangles, exhibited both delayed and reduced mortality. In particular, animals vaccinated with ORF-2 (p₂ 90) exhibited no more than 5% mortality out to day 32.

### Cumulative Mortality as Defined by RPS₆₀

TABLE 4
Tanks A and B were set up with a random mix of vaccinated and saline
vaccinated fish.

| Vaccine | RPS₆₀ Tank A | RPS₆₀ Tank B | Mean RPS₆₀ |
|---|---|---|---|
| 90 kDa ORF 1 | 96.8 | 100.0 | 98.4 |
| (^{Ps}p₁ 90) | | | |
| 90 kDa ORF 2 (p₂ | 50.0 | 77.1 | 63.6 |
| 90) | | | |
| Adjuvant + PBS | 2.0 | 11.7 | 6.9 |

In order for the test results to be acceptable, mortalities in the control group must reach 60% within 30 days after the first specific mortality has been recorded. As the results met these criteria the results were accepted as valid.

The results therefore demonstrate that both the 90 kDa ORF 1 antigen (p₁90) and the 90 kDa ORF 2 antigen (p₂ 90) are capable to provide significant protection in salmon against SRS caused by P. salmonis.

### Example 6

### Production Method Of VP2var or VP3 (50 Liter Scale)

The yeast strains: Pichia pastoris GS115-pPICZαB—VP2var. The Pichia expression system is used to express the IPN protein antigens [Research Corporation Technologies, Tucson, Ariz., see U.S. Pat. Nos. 4,808,537, 4,837,148, 4,879,231, the contents of which are hereby incorporated by reference in their entireties.

Preculture: A 2-liter baffled shake-flask containing 400 ml of YSG+ (see below) is inoculated with 600 μl of the above-identified yeast strain. The culture is incubated at 30° C., with an agitation of 270 rpm, during 23-25 hours. The optical density at 600 nm (OD₆₀₀ₙₘ) is >15 units (using a NOVASPEC II spectrophotometer), as set forth by Table 5, below.

TABLE 5
Composition of the Medium YSG+:

| COMPONENTS | CONCENTRATION |
|---|---|
| Yeast Extract | 6 g/l |
| Papaïc Soy Pepton | 5 g/l |
| Glycerol | 20 g/l |

Fermentation: The fermentor Braun D50 is prepared with 50 liters of growth medium (SAPPEY, see below). The fermentor is inoculated with a volume (V_{preculture}) of preculture determined by the equation:

V_{preculture}(ml)=V_{fermentor}(ml)×0.05/OD_{600preculture}

Where, V_{fermentor} is defined as the volume of the growth medium in the fermentor, and the OD_{600preculture} is the optical density determined at 600 nm of the preculture solution obtained above.

TABLE 6
Parameters for Fermentation

| PARAMETERS | SET POINTS |
|---|---|
| pH | 6* |
| Temperature | 30° C. |
| Air-flow | 80 l/min. |
| PO₂ | 30%** |
| Agitation | 400-(600) rpm |
| Pressure | 100 mbar |

*regulation with acid (HNO₃ 10%) and base (NH₄OH 12.5%)
**with an action on the agitation to maintain the PO₂ at 30% Automatic regulation of foam with SAG471.

TABLE 7
Composition of Growth Medium SAPPEY per 1 Liter:

| Components | Volumes |
|---|---|
| Base solution | 900 ml |
| Complement solution 1 | 100 ml |
| PTM1 solution | 4.76 ml |

TABLE 8
Quantities per 1 Liter of Base Solution
[The solution is autoclaved in the fermentor (20 min., 121° C.)]

| Components | Quantity |
|---|---|
| Yeast Extract | 11.11 g/l |
| Papaïc Soy Pepton | 22.22 g/l |
| Antifoam SAG471 | 0.11 ml/l |

TABLE 9
Quantities per 1 Liter of COMPLEMENT SOLUTION 1
(The solution is sterilised by filtration with a 0.22 μm pore membrane)

| Components | Quantity |
|---|---|
| K₂HPO₄ | 23 g/l |
| KH₂PO₄ | 118 g/l |
| Glycerol | 100 g/l |

TABLE 10
Quantity for 1 Liter of PTM1 SOLUTION

| Components | Quantity | |
|---|---|---|
| CuSO₄•5H₂O | 6 | g/l |
| Nal | 0.08 | g/l |
| MnSO₄•H₂O | 3 | g/l |
| Na₂MoO₄•2H₂O | 0.2 | g/l |
| H₃BO₃ | 0.02 | g/l |
| CoCl₂•6H₂O | 0.92 | g/l |
| ZnCl₂ | 20 | g/l |
| FeSO₄•7H₂O | 65 | g/l |
| d-biotine | 0.2 | g/l |
| H₂SO₄ | 5 | ml/l |

The solution is sterilized by filtration with a 0.22 μm pore membrane. The PTM1 solution must be added in the fermentor separately from the complement solution 1.

TABLE 11
Composition of “INDUCTION SOLUTIONS” per 1 Liter
(The methanol is added by sterile filtration
with a 0.22 μm pore membrane)

| Components | Volumes |
|---|---|
| Methanol 100% | 6.3 ml/l of culture |
| Yeast Extract solution | 22.5 ml/l of culture |

TABLE 12
Quantity for 1 Liter of YEAST EXTRACT SOLUTION
[This solution is autoclaved (20 min., 121° C.)]

| Components | Quantity |
|---|---|
| Yeast Extract | 222 g/l |

After 24 hours of growth, a first induction of recombinant protein expression is realized by the addition of methanol and yeast extract solution. At this moment, the OD₆₀₀ₙₘ is greater than about 10 units. After the induction the pO₂ decreases quickly. After about 1 hour, it increases slowly to saturation. A second induction is realized after 48 hours of culture in the same conditions. The OD₆₀₀ₙₘ reached is greater than about 13 units. After 72 hours of growth, the fermentor is cooled to a temperature lower than 20° C. The OD₆₀₀ₙₘ reached is greater than about 13 units.
Harvest and filling: The cells from the fermentor are then harvested. The culture is centrifuged (5000 g, 4° C., 20 min) in order to eliminate the pellets. The supernatant is aseptically filtrated with a 0.2 μm pore membrane (Sartobran P) and 2.5 liter aliquots are placed into one gallon bottles. These bottles are then stored at −20° C.

### Example 7

### AN Injectable Vaccine for SRS, IPN and Furunculosis

### Summary

One injectable vaccine of the present invention is a water-in-oil type vaccine that comprises a suspension of:
(i) two inactivated strains of Aeromonas salmonicida (MT004 and MT423),
(ii) two recombinant IPN viral proteins (VP2 and VP3) or antigenic fragments thereof, that are expressed by transformed yeast, Pichia pastoris in 0.85% p/v sterile saline, and
(iii) a suspension comprising inactivated recombinant strains of E. coli encoding p₁ 90 and/or p₂ 90, in phosphate buffered saline.

The VP2 (VP2var) recombinant proteins are expressed by transformed yeast, Pichia pastoris BCCM Accession No. HEM 20069 and/or BCCM Accession No. IHEM 20070, whereas the VP3 recombinant proteins are expressed by BCCM Accession No. HEM 20071, and/or BCCM Accession No. IHEM 20072. The oily adjuvant is MONTANIDE ISA711 and constitutes 70% of the vaccine's total volume. The formulation may contain residual amounts of formaldehyde, derived from inactivation of the cultures.

This particular vaccine is designed and recommended for administration by intraperitoneal injection, to protect against salmonid rickettsial septicaemia, infectious pancreatic necrosis and furunculosis in fish, more particularly salmonids, and even more particularly, in salmon.

### Presentation

This vaccine is presented in 500 ml high density polyethylene infusion flasks, closed with grey nitrile stoppers and having aluminium seals. The bottles and stoppers comply with the requirements of the relevant monographs of the European Pharmacopoeia (Ph. Eur). The containers are autoclaved at 121° C. for 20 minutes. The stoppers are autoclaved at 121° C. for 60 minutes.

### Production

### Production of A. salmonicida MT004 Antigen

An ampoule of lyophilized work seed is removed from storage and is reconstituted and incubated. This culture is then inoculated in 4 liters of sterile iron-deficient TSB to form the production culture, and then incubated at approximately 21.5° C. for 36-48 hours.

The resulting culture is then aseptically inoculated in 15-18 liters of sterile iron-deficient TSB. It is incubated at approximately 21.5° C. for 24 to 48 hours. Then a solution of sterile formaldehyde is added to the flasks to inactivate the culture. Each culture is mixed vigorously following the addition of the formaldehyde solution and is then transferred aseptically to a sterile storage bottle. The culture is kept at approximately 22° C. for 96-100 hours to ensure the inactivation of bacterial cultures and protease activity. The formaldehyde is neutralized by the is addition of a solution of 15% sodium metabisulfite. Neutralisation is completed in 20-24 hours at a temperature of approximately 22° C. The inactivated harvests are stored at 2-8° C. until they are required for mixing. The production of A. salmonicida MT004 antigen can also be performed as described below for MT423.

Production of A. salmonicida MT423 Antigen:

An ampoule of lyophilized work seed is removed from storage and reconstituted and incubated. This culture is then inoculated in 300 ml of sterile iron-supplemented TSB to form the production culture, and then incubated at approximately 21.5° C. for 36-48 hours.

The culture is next inoculated aseptically in 4 liters of sterile iron-supplemented TSB. It is incubated at approximately 21.5° C. for 36 to 48 hours. The culture of production seed is transferred aseptically to 150 liters of sterile iron-supplemented TSB in a fermentor and incubated at approximately 21.5° C. for 20-24 hours.

Then a solution of sterile formaldehyde is added to the culture flasks to inactivate them. Each culture is mixed vigorously following the addition of the formaldehyde solution and is transferred aseptically to a sterile storage bottle. The culture is kept at approximately 22° C. for 96-100 hours to ensure inactivation of the bacterial cultures and protease activity. The formaldehyde is neutralized by adding a solution of 15% sodium metabisulfite. Neutralization is completed in 20-24 hours at a temperature of approximately 22° C. The inactivated harvests are stored at 2-8° C. until they are required for mixing

### Production of Recombinant Proteins IPN (VP2 VAR) and IPN VP3:

Recombinant proteins IPN (VP2 VAR) and IPN VP3 are prepared and stored as described in Example 6 above.

### Production of Antigens:

The p₁ 90 and p₂ 90 antigens are prepared for the formulation of the vaccine as described in Example 4 above.

### Mixing of the Final Vaccine

Bulk antigens are mixed with the other antigen components, phosphate-buffered saline solution, and the oil component to obtain a bulk vaccine of the desired cell concentration.

The volumes of bulk antigens required (calculated on the individual concentrations of bulk antigen, the required concentrations of these in the end product and the batch size) are removed from storage. The bulk antigens are transferred to cool, sterile containers and are mixed thoroughly.

The volume of sterile saline required is calculated and transferred aseptically to the mixed bulk antigens. The antigens and saline are thoroughly mixed and the pH is adjusted to pH 7.0-7.4 with 10 M sodium hydroxide or 10 M hydrochloric acid (aqueous phase).

The weight of sterile oily phase required is calculated and transferred aseptically to a sterile mixing container. The oily and aqueous phases are emulsified for 5 minutes at approximately 3000 rpm. The emulsified mix is maintained at ambient temperature for 24 hours. The mix is placed in the final containers, with a nominal fill value of 505 ml. The stoppers are inserted aseptically and the seals are applied. Each container is labeled, packaged and stored at +2° C. to +8° C. under quarantine until released for sale. The batch size varies according to production requirements and is normally within the range of 100 liters to 1500 liters.

### Materials

The antigens are prepared as described above. In addition two strains of Aeromonas salmonicida are used, which derive from isolated naturally infected fish obtained from fish farmed in Scotland.

In spite of the fact that there is no evidence that there is any serological distinction between different strains of Aeromonas salmonicida, there is a scientific basis for including more than one strain in this vaccine. This is due to the fact that different isolated ones may be A-layer positive or negative. Considering that the presence or absence of this layer may not be directly linked to virulence, the absence of an A-layer allows greater exposure to outer membrane proteins (OMPs), and in particular, those OMPs produced only under conditions of iron restriction, as may is occur during the infection process. As a result, the production and immunological availability of the iron restriction outer membrane proteins (IROMPs) is thought to be important to the efficacy of the vaccine.

Aeromonas salmonicida (MT004):

The MT004 strain is an A-layer negative strain which is cultivated under conditions of iron restriction. Development under these conditions results in the production of specific iron restriction outer membrane proteins that stimulate the production of bacterial antibodies following intraperitoneal inoculation.

The strain was originally isolated from dying Atlantic salmon during an outbreak of furunculosis in on a salmon farm on the West Coast of Scotland in October 1985. It was passaged through tryptone soya broth six times and remained virulent to the host animal.

Aeromonas salmonicida (MT423):

The MT423 strain is an A-layer positive strain that has been cultivated in a fermentor under conditions of iron restriction. A-layer is a component of successful A. salmonicida vaccines and supplementation with iron has increased the protection afforded by the furunculosis vaccine.

The MT423 strain was isolated from sick Atlantic salmon from a salmon farm at Stirling University. It was passaged 16 times in Atlantic salmon and remained virulent to the host animal and is therefore appropriate for use as a vaccine strain.

Both strains are inactivated by exposure to formaldehyde, being in non-infecting organisms, whereas it retains its ability to stimulate an immune response in vaccinated fish.

The vaccine also contains the recombinant proteins VP2 var and VP3 of IPNV as described in Example 6 above.

Other Reagents are provided in Table 13:

TABLE 13
Reagents

| REAGENT | COMPONENTS | CHARACTERISTICS |
|---|---|---|
| Tryptone Soya | Pancreatic casein | Cow's milk from herds certified |
| Broth (TSB) | digestive enzyme | BSE free, originally from France, |
| | | but currently from New Zealand. |
| | | Porcine enzymes from France, |
| | | Italy and Holland. |
| | Soya digestive | No materials of biological origin |
| | papain | |
| | Sodium chloride | |
| | Hydrogenated | |
| | dipotassium | |
| | phosphate | |
| | Dextrose | Synthetic or of non-animal origin |
| | Purified water | Meets the requirements of the |
| | | European Pharmacopoeia. |
| Hydrochloric acid | — | Meets the requirements of the |
| (pH adjustment) | | European Pharmacopoeia. |
| Sodium | — | Meets the requirements of the |
| hydroxide | | European Pharmacopoeia. |
| (pH adjustment) | | |
| Formaldehyde | — | Meets the requirements of the |
| (Inactivator) | | European Pharmacopoeia. |
| Saline solution | Sodium chloride | Meets the requirements of the |
| (Diluent) | | European Pharmacopoeia. |
| | Purified water | Meets the requirements of the |
| | | European Pharmacopoeia. |
| Montanide | Contains oleic | EDQM Certified available |
| ISA711 | acid | |
| (Adjuvant) | | |

### Assays

Several tests are carried out to ensure that the consistency and quality of the vaccine and its components are maintained. These tests are described below.

Aeromonas salmonicida Strains MT004 and MT423:

The test methods used for both antigens are the same, except that the test for the presence of ROMPS is not used for the MT423 strain, since this is multiplied in an iron-enriched medium. In addition, the criteria used for some tests are different for each strain. For the sake of simplicity, the following test descriptions specify the criterion for each strain where it is appropriate.

### Purity Tests—Gram Stain:

Gram stain purity tests are carried out on each subculture during multiplication from seed to production culture. The test provides a rapid indication that the is cultivated organism has the hoped for microscopic appearance and that no atypical organism is present.

The test method is a simple Gram stain that uses conventional techniques and materials. Known Gram positive and negative control organisms are stained each time to confirm that staining and discoloration are appropriate. The test sample must only show small Gram negative rods.

### Purity Test and Characteristics of the Culture:

An additional purity test is carried out on each of the 20 liter complete cultures and on the culture in the final fermentor. The test confirms the purity of the culture and contributes to global identity security. A sample of the culture is grown on plates of tryptone soya agar and incubated at 22° C. for at least 48 hours, long enough for the different colonies to become visible. Plates inoculated with the test culture must exhibit only one type of bacterial colony. These colonies must be typical of Aeromonas salmonicida.

The Aeromonas salmonicida MT004 strain forms semi-translucent, round, convex, cream-colored colonies with regular edges. A red-brown pigmentation is produced which spreads through the medium after around 24 hours of culture.
The Aeromonas salmonicida MT423 strain: Semi-translucent, round, convex, cream-colored colonies with regular edges, but developing more slowly than the MT004 strain.
Identity of the culture: The identity of a given culture is confirmed in the samples on final fermentation. Identity tests are carried out on the final culture prior to inactivation to confirm that the correct organism has been cultured. It must be emphasized that none of these tests can differentiate the strains, but all contribute to the security of identifying the species. In addition to the purity tests, identity is confirmed by means of biochemical and agglutination characteristics:
Demonstration of the use of glucose without gas production.
A sample from the final culture is inoculated in peptone water containing 1% glucose and phenol red in tubes containing an inverted Durham tube. The inoculated cultures are incubated at 22° C. for 24-48 hours. The test sample must show the use of glucose, indicated by acid production, without gas being produced.
Demonstration of positive metabolism of cytochrome oxidase using commercially available impregnated filter papers:
A single colony from the purity test plate (culture characteristics) is spread over the filter paper. A positive result is indicated by the development of a pinkish purple pigment while a negative result is indicated by no color change. The cultures must generate a pinkish purple coloration on the test paper, indicating positive cytochrome oxidase metabolism.
Lattes cover-glass test using a diagnostic kit of pathogens from commercial fish (Bionor MONO-AS—Code DD020).
A single colony from the purity test plate (culture characteristics) is mixed with a drop of antiserum on a microscope slide. The test uses a control culture is likewise mixed with a drop of antiserum. Positive agglutination must be observed with the test sample. The negative control sample must not show any agglutination.

### Optical Density:

Optical density measurements at 580 nm are recorded at the end of each culture in 20 liter bottles and at intervals throughout final fermentation. Optical density measurements are taken from 20 liter culture bottles to ensure that each of these inoculants has grown satisfactorily. Optical density measurements are recorded at intervals throughout final fermentation to determine the optimum time for harvest, as indicated at the end of the exponential growth phase.

A sample of the culture is placed in a cuvette and the optical density is measured directly using a spectrophotometer. If necessary, the sample may be diluted in 0.85% sterile saline solution in order to obtain opacity within the spectrophotometer's range. The method is only used to confirm satisfactory growth of the inoculant and to determine the optimum time for harvest of the final fermentation. The final optical density reading is not critical and no set criterion applies. However, the final value obtained from the culture in the fermentor is normally within the following range:
8-11 for MT004 strain (without iron)
13-18 for MT423 strain (iron supplemented)

The absolute criterion for optical density is not appropriate for several reasons. First, considering that the medium used is of biological origin, there is inevitably a variation in the degree to which a specific batch will support growth. Second, the frequency of sampling for optical density is restricted to 45 minute intervals due to the need to re-sterilize the sampling port. Consequently, the precise harvest time may allow the culture to be maintained in the stationary phase for a short period of time, during which a reduction in optical density may be observed.

### Viable Count

A sample of the culture is taken for the viable count at the end of fermentation and prior to adding the inactivator. The viable count serves as a definitive measurement of yield and forms the basis for subsequent mixing of the vaccine. The viable count is carried out using the Miles and Misra method [see e.g., Hedges, Int J Food Microbiol. 25:76(3):207-14 (2002)] with Tryptone Soya Broth as diluent and Tryptone Soya Agar as growth medium. Suitable ten-fold serial dilutions of the sample are prepared and ten replicate 0.025 ml drops of each dilution placed on the agar plate. The plates are incubated at 22° C. for 24-48 hours. Only those dilutions where colonies may be clearly counted are used to calculate the viable count.

The viable count is used as the basis for mixing the vaccine. The actual count is not critical and no set criterion is applied. However, normal counts are within the range 0.3-1.5×10¹⁰/ml for both strains MT004 and MT423. The absolute criterion is not appropriate for several reasons. First, considering that the medium is used is of biological origin, there is inevitably a variation in the degree to which a specific batch will support growth.

Second, the frequency of sampling for optical density is restricted to 45 minute intervals due to the need to re-sterilize the sampling port. Consequently, the precise harvest time may allow the culture to be maintained in the stationary phase for a short period of time, during which a reduction in optical density may be observed.

### Protease Test

The protease test is carried out on a sample of material taken immediately following the inactivation period, but before the addition of sodium thiosulphate. With the improved control of the culture's conditions, no release of protease has been observed. However, because it is possible to sample the culture from the final fermentor at intervals of no less than 45 minutes, there is the possibility that some cells will die, and consequently lysis may occur prior to inactivation. This test provides the reassurance that any protease that may be released is completely inactivated.

### Protease Assay.

3 ml of inactivated culture is added to 20 mg of SKY BLUE powder suspended in 2.5 ml of PBS and incubated for 15 minutes at ambient temperature. A positive control in which 20 mg of trypsin replaces the test samples is also incubated. The SKY BLUE powder is insoluble in PBS, but if protease activity is present, the material degrades and blue dye is released into the solution. The positive control must show a blue color while negative controls must remain colorless. To be acceptable, the test samples must not exhibit any protease activity. Positive samples must show a blue coloration.

### Inactivation Test

A specific test for inactivation of the culture is carried out following neutralization of the residual inactivator. A subsequent test for continuous and complete inactivation is carried out on the mixed aqueous phase of the vaccine. The test confirms the complete, satisfactory inactivation of all viable organisms.

### Inactivation Assay:

1 ml of inactivated culture is inoculated in each of six tubes containing 9 ml of TSB. Two of these inoculated tubes are inoculated with 0.1 ml of positive control culture with Aeromonas salmonicida of the same strains as the sample being tested, inoculating with a designated concentration of between 1 and 10 organisms. Two further inoculated tubes are additionally inoculated with 0.1 ml using the same positive control culture diluted 1 in 10. Also 0.1 ml of both positive control preparations are inoculated in two tubes, each containing 9.9 ml of TSB and another two tubes of TSB medium are kept only as negative controls. Therefore, duplicates of the following tubes are prepared (a total of 12 tubes in all):
Inoculated with 1 ml of test sample
Inoculated with 1 ml of test sample+0.1 ml positive control
Inoculated with 1 ml of test sample+0.1 ml positive control (diluted 1/10)
Inoculated with 0.1 ml positive control
Inoculated with 0.1 ml positive control diluted 1/10
Not inoculated

All of the above tubes are incubated for 48 hours at 22° C. At the end of this time, any tube in which growth cannot be seen is subcultivated. Subcultivation is carried out by spreading 1 ml of the medium onto each of two plates of tryptone soya agar. The medium is left to absorb into the agar for 1 hour at ambient temperature and the plates are incubated (inverted) for 48 hours at 22° C. The original tubes are also incubated for 48 hours at 22° C.

At the end of this time, growth (or absence of growth) is recorded in all cultures. The criterion of being acceptable is that all the tubes inoculated with the test sample only and all plates inoculated from these must not show any growth. In addition, all tubes inoculated with the highest concentration of organisms of the positive control and/or all plates inoculated from these must show growth of the control organism. If the tubes inoculated exclusively with the lowest dilution of the is positive control culture and/or the plates inoculated from these show growth, similar results must be observed for the tubes and plates inoculated with the test sample plus the diluted positive control. The control mediums must remain negative.

### Test for IROMPS

This test only applies to the material of strain MT004 and applies to a sample of final bulk antigen following inactivation and neutralization but, prior to distributing the material between the storage containers. The test is a qualitative method for confirming the presence of typical iron-restricted proteins in the preparation.

SDS-PAGE electrophoresis is performed on the sample. The SDS-PAGE gels are electroblotted to PVDF membranes that are then incubated with a rat monoclonal antibody against IROMP. Coupling of the monoclonal antibody is detected by a conjugate of goat anti-rat alkaline phosphatase and displayed using a NBT-BCIP substrate. A positive control preparation of Aeromonas salmonicida IROMP is spread on the same gel together with the molecular weight markers. The method is qualitative, but the acceptance criterion requires that the samples exhibit bands consistent with those of the control preparation. More particularly, protein bands must be detected at about 70, 72, 77 and 82 kilodaltons.

### Sterility

The sterility of each container of final bulk antigen is confirmed using a specific sterility test although the inactivation test also provides additional evidence of sterility of the bulk product prior to distribution. The test provides the assurance that each container of bulk antigen is sterile.

The method used is that indicated in the Ph. Eur. Using direct inoculation thioglycollate and soya broths are incubated at 32° C. and 22° C. respectively, and both are subcultivated after 14 days of incubation. The subcultures are incubated for 7 days, while the original cultures are incubated for a total of 21 days. The method includes positive control cultures specified in the Ph. Eur.

To be acceptable the samples being tested must be sterile. The positive is control cultures must show profuse early growth (within 3 days).

TABLE 14
SEQUENCES

| SEQ | |
|---|---|
| ID NO.: | DESCRIPTION |
| 1 | Nucleotide sequence encoding the genomic 1057 ORF 1 gene. |
| 2 | Amino acid sequence expressed by the ORF 1 gene [p₁90]. |
| 3 | Nucleotide sequence encoding the genomic 1057 ORF 2 gene. |
| 4 | Amino acid sequence expressed by the ORF 2 gene [p₂90]. |
| 5 | Nucleotide sequence encoding the PEGT1 ORF 1 gene. |
| 6 | Nucleotide sequence encoding the PEGT1 ORF 2 gene. |
| 7 | Amino acid sequence of the 45 kDa protein. |
| 8 | Amino acid sequence of the 45 kDa protein minus the signal |
| | peptide. |
| 9 | Amino acid sequence of an AMP binding enzyme homolog. |
| 10 | Amino acid sequence of ORF A. |
| 11 | Amino acid sequence of ORF B. |
| 12 | Amino acid sequence of a DDE endonuclease homolog. |
| 13 | Amino acid sequence of a transposase homolog. |
| 14 | Amino acid sequence of an HlyD homolog. |
| 15 | Amino acid sequence of an AcrB/AcrD/AcrF homolog. |
| 16 | 2,092 nucleotide nucleotide sequence comprising the coding |
| | sequence of the 45 kDa protein. |

What is claimed is:

1. A p₁90 protein that has an amino acid sequence comprising at least 75% identity with the amino acid sequence of SEQ ID NO: 2;
wherein said protein is in a form selected from the group consisting of isolated, recombinant, or both isolated and recombinant.

2. The protein of claim 1 wherein the amino acid sequence is selected from the group consisting of SEQ ID NO: 2 and SEQ ID NO: 2 comprising one or more conservative amino acid substitutions.

3. An antigenic fragment of the p₁90 protein that has the amino acid sequence of SEQ ID NO: 2.

4. A chimeric polypeptide that comprises the antigenic fragment of claim 3.

5. An antibody elicited by the p₁90 protein of claim 1, or elicited by an antigenic fragment of said protein.

6. A nucleic acid that encodes the p₁90 protein of claim 1; wherein said nucleic acid is in a form selected from the group consisting of isolated, recombinant, or both isolated and recombinant.

7. The nucleic acid of claim 6 that comprises a nucleotide sequence selected from the group consisting of the nucleotide sequence of SEQ ID NO: 1, a nucleotide sequence of a DNA molecule that hybridizes under stringent conditions with the nucleic acid having the nucleotide sequence of SEQ ID NO: 1, and the nucleotide sequence of SEQ ID NO: 5.

8. An expression vector comprising the nucleic acid of claim 7.

9. The expression vector of claim 8 that is the EGT1 plasmid having the BCCM accession No. LMBP 5690.

10. A host cell that comprises the expression vector of claim 8.

11. A method for producing a recombinant p₁90 protein comprising culturing the host cell of claim 10 in a culture medium.

12. The method of claim 11 that further comprises isolating the p₁90 protein.

13. The method of claim 12 wherein the host cell is an E. coli cell.

14. A p₂90 protein that has an amino acid sequence comprising at least 75% identity with the amino acid sequence of SEQ ID NO: 4;
is wherein said protein is in a form selected from the group consisting of isolated, recombinant, or both isolated and recombinant.

15. The protein of claim 14 wherein the amino acid sequence is selected from the group consisting of SEQ ID NO: 4 and SEQ ID NO: 4 comprising one or more conservative amino acid substitutions.

16. An antigenic fragment of the p₂90 protein that has the amino acid sequence of SEQ ID NO: 4.

17. A chimeric polypeptide that comprises the antigenic fragment of claim 16.

18. An antibody elicited by the p₂90 protein of claim 14, or elicited by an antigenic fragment of said protein.

19. A nucleic acid that encodes the p₂90 protein of claim 14; wherein said nucleic acid is in a form selected from the group consisting of isolated, recombinant, or both isolated and recombinant.

20. The nucleic acid of claim 19 that comprises a nucleotide sequence selected from the group consisting of the nucleotide sequence of SEQ ID NO: 3, a nucleotide sequence of a DNA molecule that hybridizes under stringent conditions with the nucleic acid having the nucleotide sequence of SEQ ID NO: 3, and the nucleotide sequence of SEQ ID NO: 6.

21. An expression vector comprising the nucleic acid of claim 20.

22. The expression vector of claim 21 that is the EGT1 plasmid having the BCCM accession No. LMBP 5691.

23. A host cell that comprises the expression vector of claim 21.

24. A method for producing a recombinant p₂90 protein comprising culturing the host cell of claim 23 in a culture medium.

25. The method of claim 24 that further comprises isolating the p₂90 protein.

26. The method of claim 25 wherein the host cell is an E. coli cell.

27. A vaccine that comprises an antigenically effective amount of a protein selected from the group consisting of a p₁90 protein that has an amino acid sequence comprising at least 70% identity with the amino acid sequence of SEQ ID NO: 2, a p₂90 protein that has an amino acid sequence comprising at least 70% identity with the amino acid sequence of SEQ ID NO: 4, and a mixture of said p₁90 protein and said p₂90 protein.

28. The vaccine of claim 27 that further comprises a 45 protein or antigenic fragment thereof.

29. The vaccine of claim 27 that further comprises a bacterin comprised of a Yersinia ruckeri cell selected from the group consisting of BCCM accession No. of LMG P-22044, BCCM accession No. LMG P-22511, and combinations thereof.

30. The vaccine of any of claims 27 that further comprising an antigen obtained from an Infectious Pancreatic Necrosis (IPN) virus.

31. The vaccine of claim 30 wherein the antigen obtained from the IPN virus is selected from the group consisting of the VP2 var protein and the VP3 protein.

32. The vaccine of claim 27 further comprising both the VP2 var protein and the VP3 protein from Infectious Pancreatic Necrosis (IPN) virus.

33. The vaccine of claim 32 wherein the VP2 var protein is obtained from a is transformed Pichia pastoris cell, BCCM Accession No. IHEM 20069 and the VP3 protein is obtained from a transformed Pichia pastoris cell, BCCM Accession No. IHEM 20071.

34. The vaccine of claim 33 wherein the VP2 var protein is obtained from a transformed Pichia pastoris cell, BCCM Accession No. IHEM 20070 and the VP3 protein is obtained from a transformed Pichia pastoris cell, BCCM Accession No. IHEM 20072.

35. The vaccine claim 27 that further comprises an antigen obtained from Aeromonas salmonicida.

36. A method of protecting a fish from salmonid rickettsial septicemia comprising administering to the fish the vaccine of claim 27.

37. The method of claim 36 wherein the fish is a teleost.

38. The method of claim 37 wherein the teleost is a salmonid.

39. A method of protecting a fish from salmonid rickettsial septicemia and Infectious Pancreatic Necrosis comprising administering the vaccine of claim 33 to the fish.

40. The method of claim 36 wherein the fish is a salmonid.

41. The method of claim 40 wherein the salmonid is selected from the group consisting of a Salmo salar, an Oncorhynchus kisutch and an Oncorhynchus mykiss.


SEQUENCE LISTING (16 sequences)

SEQ ID NO: 1 - DNA, 2820 bp, Unknown
  The source is likely to be mycoplasma.
atgaaaaagataattacaatgatgttattggtgttatcacttgtgttggtcgcttgtaccccaagtgaagaaccaccaactacagtgccagatgttgaatccatcgaatttaatatgacttcaacgactgtagcaccaggtgaacatacactagttgcaaaagcattacctgaaggatctaatcaacaaattagatttagtattcaaggtattgtatctggtgtatccattacgggtgataagttaaatgttggtaatgctgttgaagatggtatgaaatttacagtcgtagcaacatctgtatatgatccaacaattcgtgcaacactagagtttacagttgtaaatgttggtgttgaagttgttgaaattagaacagaagaagaactacgtgcaattcatacaaatgaaggtggtttatcattatcttatgtattaatgaatgatattgaactaacagctccatggacaccaattggtattgctgaagttgaaactgattctgggcaaatcattccaggtacgccatttaatggtatctttaatggaaatggttttacaattagtggcatattagttgaaagtgaagaaccattatttaatgcaggattctttgctcaaattggtgcaactgcaattgttaagaatacaacatttgaaggtatagtaaatgcaaccggatggtccggtggtatcgcaggtattaacgaaggtttaatagaaaatgttgtatctaatgtaagagttactgtaacaggtacatccgcaggttcgctagtatctgttaatagaggtttaatccaatatgcttacggtattggtaaagttgttagtgaaacaaaccctaatacatcaggtcgttctgctggtttagtggttgctaatgatggatcaatgattgaagtgtatggtgactatcaagcacttggaacacctaactatacagcatttagtccatcaacaaacccaatgtatatgttacctacagtagatatgaaaacatcttcaacttgggcttcatttgatgcagatgtttggtatattgaaaatggtacatatccattattaaaacatgaaggattcgttccaccagtgatcgttcctgaattaggtattacaattaaaaatactgagttaaatcatgatgttgaagtatcaagtgaactacaaataaatgcagaagtcattaacccagaaggtagtgaagttattgtttatgcacttaaagaagcagtagcaggtgtagcaattagtgaaacaggtttagttacatttgatatcactacaattgctgctaacttctcatttacagtagtagtgacaattgatggtactgaagttagtgctgaaaaaacatttacaggcgtatataaccctgaaattgtagatgatacagtgtatattgaaacagaaacacaattattaaacttacttgctggacaaacaaacccagacaatttaagtaaaacatttgtattattaaatgatattgtcttaacttctaattggacagcaattggtattgcaccaaatgaagacgaaggtattgtaggtgttccatttacaggtgtatttgatggtcaaggctataagatctcaggtattagtatgccaggtggtggatggaataaaggtttctttggatatattggaacaactggtgttgttaaaaacacacactttgaaggtaatctagaagcaaacgcatggtcaggtgcacttgcagcaaataactcaggtactattcaagatgtagttgttgatattgaagtatatgtctggggtaataatggtggcgcaatcgttgaacataaccatggtctacttaaaaatattgtcgtattaggtaaagctgtatcagatagtggtcctacagcagttggactggttgttactaactttggtactttagaaaatgtatttgctaacgcagatacagtaggtacagcaaacttagtatctaatggtgctctcgctgatgatggtaaacacattattagtgcccaagactttgttaaagctacaacttatgcaaactttgatagcgcaatctggttaatcgtagatggccaagtacctgtattaattaatgaagatacagtattacctgaaacagtagtttatattgaaacagaagcagaattattaagcctacttgctggtcaagtagatccagaagcattatcaaaaacatacaaacttaaaaatgatatcgttctaacttctaattggacagcaattggtattgcaccaaatgaagacgaaggtattgtaggtgttccatttacaggtgtatttgatggtcaaggctataagatctcaggtattagtatgccaggtggcggatggaataaaggtttctttggttacattggaacaacgggtgttgttaaaaatacacactttgaaggtaatattgaagcaaacgcatggtcaggtgcacttgcagcaaataactcgggtactattatggatgttgtagtagacattgaagtatatgtctggggtaataatggtggtgcaattgttgaacataaccacggtttacttaaaaatattatcgtcttaggtaaagctgtatcagatggtggtcctacagtagttggactagttgttactaactttggtacactagaagatgtatatgcaaatgttgacacagtaggtactttaaacttagtatcatttggtagcgtagcagatgatggtacacacattattagtgcttcaaactttgttaaagcagaaacttatgcaaacttctcaagtgatgtttggacaattattgatggtagcacccctgtattaaaacaagcataa

SEQ ID NO: 2 - protein, 939 aa, Unknown
  The source is likely to be mycoplasma.
MKKIITMMLLVLSLVLVACTPSEEPPTTVPDVESIEFNMTSTTVAPGEHTLVAKALPEGSNQQIRFSIQGIVSGVSITGDKLNVGNAVEDGMKFTVVATSVYDPTIRATLEFTVVNVGVEVVEIRTEEELRAIHTNEGGLSLSYVLMNDIELTAPWTPIGIAEVETDSGQIIPGTPFNGIFNGNGFTISGILVESEEPLFNAGFFAQIGATAIVKNTTFEGIVNATGWSGGIAGINEGLIENVVSNVRVTVTGTSAGSLVSVNRGLIQYAYGIGKVVSETNPNTSGRSAGLVVANDGSMIEVYGDYQALGTPNYTAFSPSTNPMYMLPTVDMKTSSTWASFDADVWYIENGTYPLLKHEGFVPPVIVPELGITIKNTELNHDVEVSSELQINAEVINPEGSEVIVYALKEAVAGVAISETGLVTFDITTIAANFSFTVVVTIDGTEVSAEKTFTGVYNPEIVDDTVYIETETQLLNLLAGQTNPDNLSKTFVLLNDIVLTSNWTAIGIAPNEDEGIVGVPFTGVFDGQGYKISGISMPGGGWNKGFFGYIGTTGVVKNTHFEGNLEANAWSGALAANNSGTIQDVVVDIEVYVWGNNGGAIVEHNHGLLKNIVVLGKAVSDSGPTAVGLVVTNFGTLENVFANADTVGTANLVSNGALADDGKHIISAQDFVKATTYANFDSAIWLIVDGQVPVLINEDTVLPETVVYIETEAELLSLLAGQVDPEALSKTYKLKNDIVLTSNWTAIGIAPNEDEGIVGVPFTGVFDGQGYKISGISMPGGGWNKGFFGYIGTTGVVKNTHFEGNIEANAWSGALAANNSGTIMDVVVDIEVYVWGNNGGAIVEHNHGLLKNIIVLGKAVSDGGPTVVGLVVTNFGTLEDVYANVDTVGTLNLVSFGSVADDGTHIISASNFVKAETYANFSSDVWTIIDGSTPVLKQA

SEQ ID NO: 3 - DNA, 2499 bp, Unknown
  The source is likely to be mycoplasma.
atgcatttggtgttgttgctaaaaagtaaaaaggacaaatatatgaaaaaaataagcttaataatgatttttctgctttctatcctattggtaagttgtgtagaaaaagaagaaccaaaatttgatccagataaatatctagatttagagaatattgtatttgatgattttgataacggaattgacccgaatatgtgggttattggtaatagtaagtggggtgtaggtaatggtggtgtcatctatgaaaatgtccattacacaaatgacggtattgtagttcttcaaaccaatggtgacttgtatgatggtccacttcgcggtattggtaatacccatggcagacgtacaggtgcaatgattacaacaagagaagcactaggtcctggtagatttgaagtacgtatgcgtattatgccacgttttggttcaactactgctatgtggacttactattatgataatggtatgaaccatgaaatagatatcgaaagtaacgttgaaaatgactttagaaaagtatggactacaaactggattagtttaacagaatatagtactgtgtctaataccttagattttgcacaaaatgattttgaatggcgtacataccgttttgactggtttacagatccaaaacgcattgattattatattgatgaagtattagtttcatcacaatcttcttatgtaccagatcatgcaggagaatttaatattggtaattggtttccagatgcttgggcaggtgtacctgattttgaaacagactatacctatgtagactggttcaaatatacaccatttaaagaacaaccatatacaccaacaccggcaaataatcaaagtcctgcaaacttctatccatcagaaccaattgaacatccaatagcaaacctcatttcaaatgcaggttttgaaacagatgctccagcttggcgttatcctgtaactagtggtgtggaactactagaaggtgaaggtttaaacggatcaagaggaatctttgttccacaaaatgatattgcatatcaatttgtcacaggattagatgaaacctttgaaatgacatttagtgcacatgcaaaactacctttaaatggtagtggatatgttttattagagttctacccagcagagacacaaaaaattgatcagtatatgattgagtttaactcaagcgatgaagattttatagcagatacattctatggtaaagaatttacctttaatgtacctctaggaactaaacgtgttgaagtgtctttaattggaggagattctggtatatacttcgatgatttattctttaacctaactaaaaaacccaggcccgaaattgtagaagaaggtgatgatgtgcaacgtttaaacatagattttaaaaatggtattgactccaatgtttgggcagttgcaaatcaacgttggggaggtacacatcatggtggtgtaatcttccaaaacgtacactacacagaagaaggtaatttactcattcaagccaatggtgattactatgaaggtccattaaaaggtgttgaacaaaataatggaaaacgcactgggggagctatctatactaaagaagcatttggcccaggatcttttgaagtaaaagctaaaatcatgccacgttttggggcaacaacagcattttggacatttaactacttagatggtattaatagtgaaattgattttgagtttaacgtaggcaatgattttagtacagtttggttaaccaactggttaaccgaaacaaactataacaactacacccatcaaatggatagtttccataatgatggaaactggcatatataccgttttgaatggcatacactaccgacgcctcatattaaatactttatcgatggcaaacttgcatatacagaacatactaaagttccaacgatgtctgcaagatactggattggtgtatggtttccaaataactgggcaggagatccaaactttgaaacagattatttagaagttgaatatttcaaatatgagtcattcccggatcatccgtatgttgttggtccaactggggcatcctctccaacagcattttacccaacagcgccaataaaaaaaccagtttctaaccttttaccacacggtaatctagattatgaaacaggttatatgttaacaggggatgcagtgatttcaaatggtgaattgaaaactggtttactaggcagtgctgagtctcttattacagggttaaatgatgcctttgaacttacattaaagcttaaagcaaaagcctcaaataacgcaaccgtgcgcattgagtatttagataaggatttaaatgtgataagtggtgaagatattattgtatcaaacttaaacgcgaatacatttacaaactttacatccgtaattaatctagtggaaggcactagagccatcaatgtgatttttgagggaacaaatatcacatatgatgacttatttataaatttaacacacaaggtgaattga

SEQ ID NO: 4 - protein, 832 aa, Unknown
  The source is likely to be mycoplasma.
MHLVLLLKSKKDKYMKKISLIMIFLLSILLVSCVEKEEPKFDPDKYLDLENIVFDDFDNGIDPNMWVIGNSKWGVGNGGVIYENVHYTNDGIVVLQTNGDLYDGPLRGIGNTHGRRTGAMITTREALGPGRFEVRMRIMPRFGSTTAMWTYYYDNGMNHEIDIESNVENDFRKVWTTNWISLTEYSTVSNTLDFAQNDFEWRTYRFDWFTDPKRIDYYIDEVLVSSQSSYVPDHAGEFNIGNWFPDAWAGVPDFETDYTYVDWFKYTPFKEQPYTPTPANNQSPANFYPSEPIEHPIANLISNAGFETDAPAWRYPVTSGVELLEGEGLNGSRGIFVPQNDIAYQFVTGLDETFEMTFSAHAKLPLNGSGYVLLEFYPAETQKIDQYMIEFNSSDEDFIADTFYGKEFTFNVPLGTKRVEVSLIGGDSGIYFDDLFFNLTKKPRPEIVEEGDDVQRLNIDFKNGIDSNVWAVANQRWGGTHHGGVIFQNVHYTEEGNLLIQANGDYYEGPLKGVEQNNGKRTGGAIYTKEAFGPGSFEVKAKIMPRFGATTAFWTFNYLDGINSEIDFEFNVGNDFSTVWLTNWLTETNYNNYTHQMDSFHNDGNWHIYRFEWHTLPTPHIKYFIDGKLAYTEHTKVPTMSARYWIGVWFPNNWAGDPNFETDYLEVEYFKYESFPDHPYVVGPTGASSPTAFYPTAPIKKPVSNLLPHGNLDYETGYMLTGDAVISNGELKTGLLGSAESLITGLNDAFELTLKLKAKASNNATVRIEYLDKDLNVISGEDIIVSNLNANTFTNFTSVINLVEGTRAINVIFEGTNITYDDLFINLTHKVN

SEQ ID NO: 5 - DNA, 2820 bp, Artificial Sequence
  EGT optimized nucleotide sequence
atgaaaaaaatcatcaccatgatgctgctggttctgagcctggttctggtggcgtgtaccccgtctgaagaaccgccgaccaccgttccggatgtggaaagcattgaatttaacatgaccagcaccaccgtggcaccgggcgaacataccctggtggcgaaagcgctgccggaaggcagcaaccagcagattcgttttagcattcagggcattgtgagcggcgtgagcattaccggcgataaactgaacgtgggcaacgccgtggaagatggcatgaaatttaccgttgtggcgaccagcgtgtatgacccgaccattcgtgccaccctggaatttaccgtggttaacgttggcgtggaagtggtggaaattcgtaccgaagaagaactgcgcgcgattcataccaacgaaggcggcctgagcctgagctatgtgctgatgaacgatattgaactgaccgccccgtggaccccgattggcattgccgaagtggaaaccgatagcggccagattattccgggcaccccgtttaacggcatttttaacggcaacggctttaccattagcggcattctggtggaaagcgaagaaccgctgtttaacgccggcttttttgcccagattggcgccaccgccattgtgaaaaacaccacctttgaaggcattgtgaacgccaccggctggagcggcggcattgccggcattaacgaaggcctgattgaaaacgttgttagcaacgttcgtgtgaccgtgaccggcaccagcgccggtagcctggtgagcgtgaaccgtggcctgattcagtatgcctatggcattggcaaagtggtgagcgaaaccaacccgaacaccagcggtcgtagcgccggtctggtggtggcgaacgatggcagcatgattgaagtgtatggcgattatcaggcgctgggcaccccgaactataccgcctttagcccgagcaccaacccgatgtatatgctgccgaccgtggatatgaaaaccagcagcacctgggcgagctttgatgccgatgtgtggtatatcgaaaacggcacctatccgctgctgaaacatgaaggctttgtgccgccggttattgtgccggaactgggcattaccattaaaaacaccgaactgaaccatgatgtggaagtgagcagcgaactgcagattaacgccgaagtgattaacccggaaggtagcgaagttattgtttatgccctgaaagaagcggtggcgggcgttgccattagcgaaaccggcctggtgacctttgatattaccaccattgcggcgaactttagctttaccgtggtggtgaccattgatggcaccgaagtgagcgccgaaaaaacctttaccggcgtgtataacccggaaattgtggatgataccgtctatatcgaaaccgaaacccagctgctgaacctgctggcgggccagaccaacccggataacctgagcaaaacctttgtgctgctgaatgacattgtgctgaccagcaactggaccgccattggtattgccccgaacgaagatgaaggtattgttggcgttccgtttaccggtgtgtttgatggccagggctacaaaattagcggtattagcatgccgggtggcggctggaacaaaggcttttttggctatatcggcaccaccggcgtggtgaaaaatacccatttcgaaggtaacctggaagcgaacgcctggtctggcgccctggcggcgaacaacagcggcaccattcaggatgtggtggtggatatcgaagtgtatgtttggggcaacaacggcggtgccattgtggaacataaccatggcctgctgaaaaacattgtggtgctgggtaaagcggtgagcgatagcggtccgaccgccgtgggtctggtggttaccaactttggcaccctggaaaacgtgtttgccaacgccgataccgtgggcaccgccaacctggtgagcaacggtgccctggcggatgatggcaaacacattatcagcgcccaggattttgtgaaagcgaccacctatgccaactttgatagcgccatttggctgattgtggatggccaggtgccggttctgattaacgaagataccgtgctgccggaaaccgtggtgtatattgaaaccgaagcggaactgctgtctctgctggcgggtcaggtggatccggaagcgctgtctaaaacctacaaactgaaaaacgatatcgtgctgacctctaactggacggcgatcggcatcgctccgaatgaagatgagggcatcgtcggcgtcccgttcaccggcgtgttcgacggtcagggttataaaatttctggcatttctatgccgggtggtggttggaataaaggtttcttcggttacattggcaccaccggtgttgttaaaaacactcactttgagggtaatattgaagcgaatgcctggagcggcgctctggccgccaacaactctggcaccattatggatgttgttgtcgatattgaagtttacgtgtggggcaataatggtggcgccatcgttgaacacaatcacggtctgctgaaaaatatcattgttctgggtaaagccgtttctgatggcggtccgacggtggtgggcctggttgtgacgaatttcggcacgctggaagatgtgtatgccaatgttgataccgttggcaccctgaatctggtgagctttggcagcgtggccgatgatggcacccatatcattagcgccagcaactttgttaaagcggaaacctatgccaattttagcagcgatgtgtggaccattattgatggcagcaccccggtgctgaaacaggcgtaa

SEQ ID NO: 6 - DNA, 2509 bp, Artificial Sequence
  EGT optimized nucleotide sequence
atgcatctggtgctgctgctgaaaagcaaaaaagataaatacatgaaaaaaatcagcctgatcatgatttttctgctgtctattctgctggtgagctgtgtggaaaaagaagaaccgaaattcgatccggataaatacctggatctggaaaacatcgttttcgatgatttcgataacggcattgatccgaacatgtgggtgattggcaacagcaaatggggcgtgggcaacggcggcgtgatttatgaaaacgtccattacaccaacgatggcattgtggtgctgcagaccaacggcgatctgtatgatggcccgctgcgtggcattggcaacacccatggccgtcgtaccggcgccatgattaccacccgtgaagcgctgggtccgggccgttttgaagttcgtatgcgcattatgccgcgttttggcagcaccaccgccatgtggacctattattatgataacggcatgaaccacgaaattgatatcgaaagcaacgtggaaaacgattttcgtaaagtttggaccaccaactggatcagcctgaccgaatatagcaccgtgagcaacaccctggattttgcccagaacgattttgaatggcgtacctatcgttttgattggtttaccgatccgaaacgtatcgattactacattgatgaagtgctggtgagcagccagagcagctatgtgccggatcatgccggcgaatttaacattggcaactggtttccggatgcctgggcaggcgttccggattttgaaaccgattatacctacgtggattggtttaaatacaccccgtttaaagaacagccgtataccccgaccccggcgaataaccagagcccggcgaacttttatccgagcgaaccgattgaacatccgattgccaacctgattagcaacgccggcttcgaaaccgatgccccggcatggcgttatccggtgaccagcggcgtggaactgctggaaggcgaaggcctgaacggcagccgtggcatttttgtgccgcagaacgatattgcctatcagtttgtgaccggcctggatgaaacctttgaaatgacctttagcgcccatgccaaactgccgctgaacggtagcggctatgtgctgctggaattttatccggcggaaacccagaaaattgaccagtatatgatcgaattcaacagcagcgatgaagattttatcgccgataccttctatggcaaagaatttacctttaacgttccgctgggcaccaaacgtgtggaagtgagcctgattggcggcgatagcggcatttattttgacgacctgttcttcaacctgaccaaaaaaccgcgtccggaaattgtggaagaaggcgacgacgttcagcgtctgaacattgatttcaaaaacggcatcgatagcaacgtgtgggcggtggcgaatcagcgttggggcggcacgcatcatggcggtgtgatttttcagaacgttcactataccgaagaaggcaacctgctgattcaggcgaacggcgattattatgaaggtccgctgaaaggcgttgaacagaacaacggcaaacgtaccggcggtgccatttataccaaagaagcgtttggcccgggtagctttgaagtgaaagcgaaaatcatgccgcgctttggtgccaccacggcgttttggacctttaactatctggatggcatcaacagcgaaatcgattttgaattcaacgtgggcaacgattttagcaccgtgtggctgaccaactggctgaccgaaaccaactataacaactacacccatcagatggatagctttcataacgatggcaactggcatatttatcgctttgaatggcataccctgccgaccccgcatattaaatacttcatcgacggcaaactggcgtataccgaacataccaaagtgccgaccatgagcgcccgttattggattggcgtgtggtttccgaacaactgggcgggtgatccgaactttgaaaccgactatctggaagtggaatacttcaaatacgaaagctttccggatcatccgtatgttgttggcccgaccggtgcctctagcccgaccgccttttatccgaccgccccgattaaaaaaccggtgagcaacctgctgccgcatggcaacctggattatgaaaccggctatatgctgaccggcgatgccgtgattagcaatggcgaactgaaaaccggcctgctgggcagcgccgaaagcctgattaccggcctgaacgatgcctttgaactgaccctgaaactgaaagcgaaagcgagcaacaacgccaccgttcgtattgaatacctggataaagatctgaacgttatcagcggcgaagatattattgtgagcaatctgaacgccaacacctttaccaactttaccagcgtgattaacctggttgaaggcacccgtgccattaacgttattttcgaaggcacgaacattacctatgatgacctgtttattaacctgacccacaaagtgaactaataagtcgacg

SEQ ID NO: 7 - protein, 438 aa, Piscirickettsia salmonis
MKVKMIVAAVAVAGLTATAANAADNGKLQLQINQLKAQHTQLQQQVANLQGQGQTTGAVHVGAVGGELISENNYDGRGLDLLKSLAKAGSNAPLLTIGGTLEADAQMNRNGNVGSGSTSGDPSGLNYTDGTSSSAFYLDTARIDILAHVNDWVNGEISYDLNGDSGLHTGSLLVGNLNQLPVYGQIGKFYPDAGLFELASDDVYSSSLVKRYFRPDAQNGASVGFYKAGLHTSLTAFKTSAPQANAANYNQATSDWSAQADYTFNAGQVNATIGAGYLSNMVNTNDSFTATGAGTGTQKDRLPMANVSAKIGFGPFEALATYAQTLKGLANTTGGTTKLKAFDLEGAYHFQAVKPMTVMLGYSRTYGFDKVGPVDQFIDGNTAITINNKKDQWLLGVNSEVFKNTTVGLEYARVGQLDSTGTDTNRYNVLTADMTVKF

SEQ ID NO: 8 - protein, 416 aa, Piscirickettsia salmonis
ADNGKLQLQINQLKAQHTQLQQQVANLQGQGQTTGAVHVGAVGGELISENNYDGRGLDLLKSLAKAGSNAPLLTIGGTLEADAQMNRNGNVGSGSTSGDPSGLNYTDGTSSSAFYLDTARIDILAHVNDWVNGEISYDLNGDSGLHTGSLLVGNLNQLPVYGQIGKFYPDAGLFELASDDVYSSSLVKRYFRPDAQNGASVGFYKAGLHTSLTAFKTSAPQANAANYNQATSDWSAQADYTFNAGQVNATIGAGYLSNMVNTNDSFTATGAGTGTQKDRLPMANVSAKIGFGPFEALATYAQTLKGLANTTGGTTKLKAFDLEGAYHFQAVKPMTVMLGYSRTYGFDKVGPVDQFIDGNTAITINNKKDQWLLGVNSEVFKNTTVGLEYARVGQLDSTGTDTNRYNVLTADMTVKF

SEQ ID NO: 9 - protein, 367 aa, Piscirickettsia salmonis
MATLAVQREVYMSDPDDIAVILYTSGTTGQPKGAMLSHRALVQNCIDLNLCWGFTDSDVLLHTLPLFHVHGLFFALHSVLYASASMILQAKFDPMEVIISLIQATVFMGVPTYYTRLLKEAEFTGSRAAQVRLFISGSAPLHEKTFQGFYQRTGKTLVERYGMSETGINTSNPLHGERKFGTVGTALEHVTVRVVDEVSEKVLMPGQTGEVQVQGRHLFSGYWQKEDQTDGAFTCDQFFKTGDLGYLDEQGYLTLVGRVKDMIISGGLNIYPKEIETAIDRVTGVNESAVVGVAHEDLGEGVVAVVVLQDNANMLAEHIIAYCKASLADFKCPKKVVFIDQLPRNTMGKVQKNQLRQQYQAIFADAH

SEQ ID NO: 10 - protein, 39 aa, Piscirickettsia salmonis
MCKIPTEVATLTAELNKRLRLNMFACLYIDFIPPCINRY

SEQ ID NO: 11 - protein, 127 aa, Piscirickettsia salmonis
MKNLIYAQRLLYFAVLIAVIVTFVQPFLMPIKLADVPLMPLVVASIYSLIFAAALALAAYKLPSKAGWPRFLLVILFIGDAMPAVKNWLVLWHTTELFAIIYLMKLMLMLAAILLSLSKLARDFYKC

SEQ ID NO: 12 - protein, 74 aa, Piscirickettsia salmonis
MDNASFHKSKGVKEAIEDAGCHLLFLPPYSPDLNPIEHVWSPLKNRVRMKLDQDEINLETALSQVMKSMSETIR

SEQ ID NO: 13 - protein, 81 aa, Piscirickettsia salmonis
MPSPYSYDLRIRALKMIDEGIPITQISKLLKISRDTLHRWKNRRDHTGDVKARFGYQTGYNHKISDMKEFQKFIDREFPGS

SEQ ID NO: 14 - protein, 319 aa, Piscirickettsia salmonis
LKSQKPPGNLTYNLSAPWSQLMALMSPRKSLARLSVFLFQSGDFVEKGRPLVQLDDRTEQANLLQYKAKLKLDQLTYDRDRSLLKKNAISRQDVDTALTSLEQTKAQMLATEVSISQKLIRAPFSGKIGIRNVNLGQYISPGTNIVSLQSINPLHVNFSLPQEDMNKIKLGQKISAHVDTFAGREFTGTITAMNSEVDSNTRTIEIQASLPNPKHELYPGMFTTVQVYLPVLPKVLTLPHTAVTYTLYGNSVYLIQLNGKKNQQGEPTGTVTRISIQTGDQRSNTVVINKGLKAGDLIVDGGQLKLENGAAIALKNTTQ

SEQ ID NO: 15 - protein, 572 aa, Piscirickettsia salmonis
MVLAIGLVVDDAIIVVENVHRHIEEGKQPFDAALIGAREIASPVIAMTITLAAVYAPIAFVGGITGALFKEFALTLAAAVIVSGVIALTLSPMMCSKLLVADNANGGLAHWLDRQFLRLQQRYERILHHTLEHRPVVLTFGLIILVGIFGMLKMTQKQLAPHEDQGFLITFASAPKYANINYVEKYSEEFAKIYKSFPAIADYFIINTTGAGTFPSQVTSGAVLKPWRDRSMTTMQLQPLLQHKLNQITGLQAQAIQMPALPGPDGMPIQFVLTSTADYSVLNNVMTKFKAAADKSGLFLFSSSDLKFNKPKLNIAIDRAKAAQMGITMQQIGSTLSTLLSGGKVNYFSLDGRSYKVIPQLADNERLTPQQLNNNYIKTAAGALIPLSTLITLSTSIEPGTLNQFQQLNSATLSAVAMPGHTDTEALNFLKAQATKLMPKGMSYNFSGQSRTLVQEGNALIYTFFFALIMIFLVLAAQFESFRDPFIIMFTVPMAIFGAAIPMAFGWTSLNIYTEIGLVTLIGLITKHGILMVQFANDLQEQEGRDIRSAIEHAAGMRLRPILMTTAAMVVG

SEQ ID NO: 16 - DNA, 2092 bp, Piscirickettsia salmonis
ccaagaactatcaaaaactatataggcaaagtataaagtctgaagcttaacctttgcttaaatgtacatcaggcttaaggtgatttctgttgagtattttcagagtcttaagctcaatttaatctttcttaaggttgaaaacaggctaaaatcaacattttgataaaattattaatttttttttattgttcttttttaatcggtttttatcctaatttgatagatagttatcgaaattcaataagttttgtttttaattgaattttttttacgagtttgggttttacaaagtgaatttacctggttatagtagccccagttgcttaatagcacttaaatgtgtatccagataaaaacaagttagggtaaaaagaatgaaagtaaaaatgattgttgcagctgtagctgttgcaggtttaacagcgactgccgcaaatgccgctgataatggtaagcttcaattacaaatcaaccaattgaaggcgcaacacactcaacttcaacagcaagttgctaatctgcaaggtcaaggccaaactactggtgccgttcacgttggcgctgttggtggtgaactaatctctgaaaataactacgatggtcgtggcttagatcttcttaaatcattagcgaaagcaggcagcaatgcaccgttattaactattggtggtacgttagaagctgatgcgcaaatgaaccgtaacggtaatgttggatctggttctacttctggtgacccttctggccttaactatactgatggaactagcagttctgcattctatttagatactgcacgtattgatatcttagcgcatgtgaatgactgggttaacggtgaaatctcgtatgacttaaatggtgatagtggtcttcacactggtagccttttagtgggtaacctcaatcaattaccagtttatggtcaaatcggtaaattctacccagatgcaggtttgtttgaattagctagtgatgatgtttattcttctagcttagtcaagcgttatttccgtccagatgcgcaaaatggtgcatctgtaggcttctataaagcaggcttacatacttctttaactgcatttaaaacgtctgctccacaagctaatgctgctaactataaccaagcaactagtgattggtctgcacaagcggattacacttttaatgcaggtcaagtcaatgccactataggtgcaggttacttatctaatatggtgaataccaatgacagcttcactgcaacaggtgcaggaactggtacacaaaaagatcggctaccgatggctaatgtaagcgctaagattggctttggtccatttgaagcccttgctacttatgctcaaacattaaaaggtttggcgaatactacaggtggtacaacgaagttgaaagcctttgatttagaaggtgcttaccacttccaagctgtgaagccgatgactgtgatgttaggttatagccgtacatatggctttgataaggttggacctgttgatcagtttattgatggtaatactgcgattactatcaataacaaaaaagaccaatggttattgggtgtaaactctgaagtatttaagaacacaacggttggtcttgagtatgcgcgtgtaggtcagcttgatagcacaggtactgacactaaccgctacaacgtattgactgcggatatgactgttaagttctaatttaagagctttaaagttttcaaaaaggcgctgcggcgcctttttttatgggcgttaattattggtaatgtaggctagtatttaaatttgtgagtgatgagagatgaaaaatttaatctatgcacagcgtttgctttattttgccgtattgattgcggtgattgtcacctttgttcagccatttctaatgccgattaagcttgctgatgtgcctttaatgccgctcgtggtcgcttcgatttattccttgatttttgctgcagctttagcattagctgcatataaattaccgagcaaagctggttggccgcggtttttgttggtgattttatttattggggatgcgatgcctgcggtaaaaaactggctagtgctttggcatacgacggagctttttgcga